(19) United States
(12) Patent Application Publication
(10) Pub. No.: US 2015/0142336 A1
(43) Pub. Date: May 21, 2015

(54) PREDICTING THE INFLUENCE OF MINERAL ADDITIONS ON REACTION AND PROPERTY DEVELOPMENT IN CEMENTITIOUS MIXTURES

(71) Applicant: The Regents of the University of California, Oakland, CA (US)

(72) Inventors: Gaurav Sant, Los Angeles, CA (US); Tandre Oey, Sunnyvale, CA (US); Aditya Kumar, Los Angeles, CA (US)

(21) Appl. No.: 14/406,181

(22) PCT Filed: Jun. 7, 2013
(86) PCT No.: PCT/US13/44687

§ 371 Date: Dec. 5, 2014

Related U.S. Application Data
(60) Provisional application No. 61/656,612, filed on Jun. 7, 2012.

(51) Int. Cl. G01N 33/38

(52) U.S. Cl. CPC G01N 33/383
USPC 702/30

(57) ABSTRACT

A mechanical property of a cementitious mixture is predicted by: (1) receiving user input characterizing a mixture of a cement and a mineral addition, the user input corresponding to at least one of: (a) a size characteristic of the cement; (b) a size characteristic of the mineral addition; and (c) a replacement level of the cement by the mineral addition in the mixture; (2) based on the user input, deriving a predicted cumulated heat released by the mixture through hydration for a reaction time period; and (3) based on the predicted cumulated heat released, deriving a predicted mechanical property of the mixture at an age corresponding to the reaction time period.

### CROSS-REFERENCE TO RELATED APPLICATION

This application claims the benefit of U.S. Provisional Application No. 61/656,612 filed on Jun. 7, 2012, the disclosure of which is incorporated herein by reference in its entirety.

### STATEMENT REGARDING FEDERALLY SPONSORED RESEARCH OR DEVELOPMENT

This invention was made with Government support of Grant No. CMMI-1066583, awarded by the National Science Foundation. The Government has certain rights in this invention.

### FIELD OF THE INVENTION

The invention generally relates to cementitious mixtures and, more particularly, to predicting the influence of mineral additions, such as those based on calcium carbonate (CaCO₃) on reaction and property development in cementitious mixtures.

### BACKGROUND

CO₂ emissions resulting from cement production are a cause of considerable concern to the concrete construction community. To reduce the CO₂ impact of cement production and use, the construction industry is making ever more substantial efforts to reduce and optimize the use of cement in concrete. Amongst these efforts are initiatives to use mineral substances to replace Portland cement in concrete. While much desired from a sustainability basis, proper concrete mixture proportioning is important to attain desired properties. For example, large reductions in the Portland cement content can be detrimental due to their role in delaying or depressing property development, and hence the ability to utilize “low cement content” concretes. Such reductions in constructability present obstacles to the commercial use and deployment of “low cement content” concretes.

In spite of advances, concrete mixture proportioning is typically an empirical approach in which a large number and combinations of materials are evaluated before a desired mixture proportion is achieved. This approach is expensive, laborious, and time-consuming. Thus, to ease the proportioning of sustainable mixtures, it would be desirable if a cement or concrete producer is able to virtually estimate the response of a mixture's binder fraction in relation to hydration and property development.

It is against this background that a need arose to develop the embodiments described herein.

### SUMMARY

One aspect of this disclosure relates to a non-transitory computer-readable storage medium. In one embodiment, the storage medium includes executable instructions to: (1) receive user input characterizing a mixture of a cement and a mineral addition, the user input corresponding to at least one of: (a) a size characteristic of the cement; (b) a size characteristic of the mineral addition; and (c) a replacement level of the cement by the mineral addition in the mixture; (2) based on the user input, derive a predicted cumulated heat released by the mixture through hydration for a reaction time period; and (3) based on the predicted cumulated heat released, derive a predicted mechanical property of the mixture at an age corresponding to the reaction time period.

In another embodiment, the storage medium includes executable instructions to: (1) provide a prediction model relating (a) a size characteristic of a cement, (b) a size characteristic of a mineral addition, (c) a replacement level of the cement by the mineral addition in a cementitious mixture, and (d) a mechanical property of the cementitious mixture; (2) receive user input corresponding to a desired value of the mechanical property; and (3) based on the prediction model, identify a candidate cementitious mixture having a predicted value of the mechanical property that matches the desired value of the mechanical property.

Other aspects and embodiments of this disclosure are also contemplated. The foregoing summary and the following detailed description are not meant to restrict this disclosure to any particular embodiment but are merely meant to describe some embodiments of this disclosure.

### BRIEF DESCRIPTION OF THE DRAWINGS

For a better understanding of the nature and objects of some embodiments of this disclosure, reference should be made to the following detailed description taken in conjunction with the accompanying drawings.

FIG. 1: Particle size distributions for: (a) cement, (b) limestone, and (c) quartz used in Example 1. The uncertainty in the measured particle size distribution is about 6%.

FIG. 2: The correlation between the level (weight) of cement replacement and the change induced in the available solid surface area in the system for: (a) limestone and (b) quartz powders. The uncertainty in the calculated area multiplier (AM) stems from the uncertainty in the particle size analysis and is correspondingly about 6%.

FIG. 3: Measured heat evolution (in terms of mW/g_{cement} versus time) profiles for binary paste systems prepared for w/s=0.45. For a given mixture, the uncertainty in the measured heat flow is about 2% based on the heat flow measured on six replicate paste specimens between 1 and 72 h.

FIG. 4: (a and b) Measured heat profiles for plain and binary pastes for corresponding AM values and (c) measured heat profiles for plain cement pastes prepared at different w/c. For a given mixture, the uncertainty in the measured heat flow is about 2% based on the heat flow measured on six replicate paste specimens between 1 and 72 h.

FIG. 5: The correlation between the AM and parameters corresponding to the measured heat flow profiles: (a) slope of the acceleration regime; (b) heat rate at the main peak; and (c) inverse of time to main peak. In all graphs, the solid line fits the linear portion of the dataset, and the dashed line projects a linear extrapolation, if a departure from linearity is noted. The thin dashed lines show a 10% bound to the best-fit line. For a given mixture, the uncertainty in the measured heat flow is about 2% based on the heat flow measured on six replicate paste specimens between 1 and 72 h.

FIG. 6: Comparison of measured and calculated (boundary nucleation and growth (BNG) model) heat profiles for paste mixtures. For a given mixture, the uncertainty in the measured heat flow is about 2% based on the heat flow measured on six replicate paste specimens between 1 and 72 h.

FIG. 7: (a) A comparison of the area factor (a_{factor}) plotted as a function of the AM for systems simulated using the BNG approach and product nuclei per gram of cement computed using the BNG approach as a function of: (b) replacement level for limestone systems (c) replacement level for quartz systems and (d) AM for limestone and quartz systems. As the calculations are deterministic for a given set of parameters, the numerical solution shows no uncertainty.

FIG. 8: A representative set of simulated and measured heat evolution profiles for paste systems. For a given mixture, the uncertainty in the measured heat flow is about 2% based on the heat flow measured on six replicate paste specimens between 1 and 72 h.

FIG. 9: Product nuclei per gram of cement computed using the multiphase reaction ensemble (MRE) approach as a function of: (a) replacement level for limestone systems, (b) replacement level for quartz systems, and (c) AM for limestone and quartz systems. As the calculations are deterministic for a given set of parameters, the numerical solution shows no uncertainty.

FIG. 10: (a) Simulations of heat released during the hydration of a single C₃S particle (5 or 15 μm) with no limestone filler (Reference), 10% replacement of limestone filler (10% Limestone) with lower energy barrier of C—S—H nucleation, and 10% replacement of quartz filler (10% Quartz) with energy barrier of C—S—H nucleation identical to C₃S and (b) simulated influence on hydration rates of carbonate anion sorption on the C—S—H. All curves represent a single simulation; multiple simulations on similar systems indicate that the reproducibility of any curve is within about 2% at any point.

FIG. 11: Particle size distributions for the: (a) cement and (b) limestone used in Example 2.

FIG. 12: Representative graphs showing reaction rates as measured using isothermal calorimetry to highlight the influence of intergrinding and post-blending for: (a) Type I/II ordinary Portland cement (OPC), and (b) 50:50 blend of Type III and Type I/II OPC and (c) Type III OPC.

FIG. 13: calorimetric parameters and best fit lines (dashed lines) as a function of the AM for: (a) slope during the acceleration period, (b) heat flow value at the main heat peak and (c) inverse of time to reach the main heat peak. The solid points are adapted from a published source, and the open symbols represent mixtures with 15% blended limestone. The horizontal dashed line indicates the calorimetric parameter value for the interground OPC-L cement.

FIG. 14: The evolution of compressive strength in interground and blended limestone (paste) systems for a variety of limestone particle sizes, for two cement types over: (a) the first day and (b and c) over the first 28 days of hydration. Except the plain cement pastes (marked REF), these systems were constituted at w/s=0.450, corresponding to w/c=0.529 for all mixtures.

FIG. 15: Compressive strength development at ages of 1, 3, 7, and 28 days of hydration as a function of the cumulative heat normalized by water content for the OPC-L, Type I/II, and Type III cements, and their limestone blended mixtures. The thick dashed line represents the (linear) best fit line with 20% bounds placed on either direction (thin dashed lines). The datapoints (adapted from Example 3) include evaluations conducted on Type I/II, Type II/V and Type III cements, for cement replacement levels ranging between 0% and 50% (weight basis) by particle size classified limestone, for strength and heat determinations carried out at 1, 3, 7, and 28 days. The compressive strength of the 50:50 OPC blends containing 15% (blended) limestone of different particle sizes was estimated using the heat release measured through hydration, and the strength-heat correlation function detailed in the figure.

FIG. 16: Representative graphs which show a comparison of the measured and BNG simulated heat release behavior for binary (OPC+limestone) paste systems.

FIG. 17: Results of the BNG calculations which describe: (a) the nucleation density as a function of the limestone particle size, and product nuclei per gram of cement as a function of: (b) the AM for the various cement types used in Example 2 and (c) effective surface area available per unit mass of cement. The datapoints (adapted from Example 3) include evaluations conducted on Type I/II, Type II/V, and Type III cements, for cement replacement levels ranging between 0 and 50% (weight basis) by particle size classified limestone.

FIG. 18: Particle size distributions for the: (a) cement and (b) limestone used in Example 3.

FIG. 19: Representative graphs showing the rate of heat release measured using isothermal calorimetry to highlight the influence of: (a) cement type, (b) limestone particle size and (c) cement replacement level on hydration reaction rates.

FIG. 20: Representative graphs showing: (a) the experimental time of initial set and (b) the calculated centroidal solid-to-solid distance functions for varying cement replacement levels. It should be noted that a plain paste for a cement replacement level of 30% has a w/c corresponding to a paste containing 30% limestone (w/c=w/s=0.643 in the first case), while w/c=0.643 and w/s=0.45 in the latter case.

FIG. 21: Generation of virtual microstructures with varying space-to-solids (water-to-solids ratios), which show the influences of solid particle sizes and individual phase fractions on the distances between particles. The 3D images shown correspond to the generation of representative elementary volumes (REVs) for w/s (weight basis) of: (a) 5.0 (b) 0.5 and (c) 0.1.

FIG. 22: Representative graphs showing strength evolution as a function of time: (a) for varying limestone particle sizes, (b) for varying cement replacement levels, (c) to compare the effects of w/c correspondence and cement replacement on the measured compressive strength. FIG. 22(d) shows the capillary porosity at different extents of hydration as a function of w/c and limestone addition (at the same w/c). The dashed vertical lines show w/c correspondence points for 10% (w/c=0.50) and 30% (w/c=0.643) replacement (weight basis) of cement by limestone for mixtures composed at w/s=0.45.

FIG. 23: The role of w/c on the: (a) hydration response and (b) strength evolution and (c) the evolution of the gel-space ratio in the system.

FIG. 24: The compressive strength as a function of cumulative heat release, normalized by the initial water content for all the mixtures evaluated in Example 3. The thick dashed line represents the (linear) best fit line with 20% bounds placed on either direction (thin lines).

FIG. 25: calorimetric parameters for cementitious (paste) mixtures as a function of AM for: (a) slope in the acceleration period, (b) heat flow at peak, (c) the inverse of time to peak and (d) the multiplication factors for each calorimetric parameter as a function of the cement fineness. The dashed lines represent the best mathematical fit to the experimental datasets.

FIG. 26: A comparison of measured and predicted parameters for mixed limestone systems for mixtures composed at a given cement replacement level for: (a) slope during the acceleration period, (b) heat flow at peak and (c) inverse of time to the main heat peak.

FIG. 27: Representative reaction curves which compare measured and calculated heat signatures for a variety of cement types and limestone sizes for: (top) heat flow and (bottom) cumulative heat release over the first 72 h. FIG. 27(a) shows the step specific use of different equations to represent different regimes of the heat flow curve.

FIG. 28: A comparison of measured and predicted values for a variety of “blind tests” for: (a) cumulative heat release normalized by water content and (b) compressive strength evolution.

FIG. 29: A computer configured in accordance with an embodiment of this disclosure.

### DETAILED DESCRIPTION

The replacement of cement by less reactive mineral additions sometimes can retard property development in cementitious mixtures. However, the replacement of cement by fine mineral fillers or other additions can accelerate hydration rates. Under certain conditions, such accelerations can act to partially offset the reduced rate of strength gain in “low cement content” concretes.

Embodiments of this disclosure provide methods, tools, and prediction models to use mineral additions as replacement materials for cement through correlations of the content of the mineral additions and their size characteristics to the extent of acceleration and development of mechanical properties, such as compressive strength or elastic modulus. Examples of mineral additions include limestone, quartz, Fly ash, silica fume, and blends or combinations of two or more of such mineral additions. Examples of cements include Portland cement, including ASTM C150 compliant ordinary Portland cements (OPCs) such as Type I OPC, Type Ia OPC, Type II OPC, Type II(MH) OPC, Type Ha OPC, Type II(MH)a OPC, Type III OPC, Type IIIa OPC, Type IV OPC, and Type V OPC, as well as blends or combinations of two or more of such OPCs, such as Type I/II OPC, Type II/V OPC, and so forth. Other examples of cements include energetically modified cements, Portland cement blends, and non-Portland hydraulic cements including calcium aluminate/sulfoaluminate cements amongst others.

Embodiments of this disclosure provide an improved and easy-to-use tool for construction technologists to develop cementitious mixtures with reduced clinker factors (for cement) and reduced cement content (for concretes), which can display comparable (and potentially superior) properties as OPC systems. Based on a prediction model, this tool can be used to predict the hydration response for a desired mixture proportion using characteristics about a cement and a mineral addition as inputs, eliminating the need for conducting laborious and time-consuming experiments. This tool provides a systematic approach to design mixture proportions, where construction technologists can dial in characteristics of mineral additions and use the tool to predict a reaction rate to extrapolate resulting mechanical properties through hydration at a particular age.

In some embodiments, a prediction model is developed to relate (a) a size characteristic of a cement, (b) a size characteristic of a mineral addition, (c) a replacement level of the cement by the mineral addition in a cementitious mixture, and (d) a mechanical property of the cementitious mixture. The size characteristic of the cement can be specified in terms of, for example, a particle size distribution of the cement, a median particle size (d₅₀) of the cement, a specific surface area of the cement, or a combination of two or more of such characteristics. Similarly, the size characteristic of the mineral addition can be specified in terms of, for example, a particle size distribution of the mineral addition, a median particle size (d₅₀) of the mineral addition, a specific surface area of the mineral addition, or a combination of two or more of such characteristics. The replacement level of the cement by the mineral addition can be specified, for example, on a mass or weight basis, such as r % by weight of the cement replaced by the mineral addition, where r can be in the range of 0 to 50, in increments of 1, 2, 3, 4, 5, 10, or other increments.

Once developed, the prediction model is incorporated in a tool to provide a variety of functionality to aid the design and development of cementitious mixtures by construction technologists. The tool can be implemented in hardware, software, or a combination of hardware and software.

In some embodiments, the tool receives user input characterizing a cementitious mixture of the cement and the mineral addition. The user input corresponds to at least one of: (a) a size characteristic of the cement; (b) a size characteristic of the mineral addition; and (c) a replacement level of the cement by the mineral addition in the cementitious mixture. The user input also can correspond to an age of the cementitious mixture through hydration for a reaction time period, such as in the range of 0 to 72 h, at 1 day, at 2 days, at 3 days, at 4 days, at 5 days, at 6 days, at 7 days, or at 28 days. A certain subset of these characteristics can be specified by the user, while a remaining subset of these characteristics can be pre-defined, pre-selected, or recommended by the tool. Based on the user input, the tool performs calculations using the prediction model to derive a predicted value of the mechanical property of the mixture at the age corresponding to the reaction time period. In some embodiments, the tool derives a predicted cumulated heat released by the cementitious mixture through hydration for the reaction time period, and then, based on the predicted cumulated heat released, the tool derives the predicted value of the mechanical property at the age corresponding to the reaction time period.

The derivation of the predicted cumulated heat released can include deriving an area multiplier (AM), which characterizes a change in solid surface area resulting from replacement of the cement by the mineral addition in the cementitious mixture. As further explained in the examples that follow, the AM can be represented using a mathematical relation involving a specific surface area of the cement, a specific surface area of the mineral addition, and a replacement level of the cement by the mineral addition. Based on the AM, the tool can derive calorimetric parameters characterizing a predicted heat flow response of the cementitious mixture through hydration. Examples of the calorimetric parameters include a slope during an acceleration time period, a heat flow at a main peak, and a time (e.g., inverse time) to reach the main peak. Once the predicted heat flow response is characterized, the tool derives the predicted cumulated heat released by summing or accumulating the predicted heat flow response over time, such as by integrating the predicted heat flow response over at least a portion of the reaction time period.

Once the predicted cumulated heat released is derived, the tool derives the predicted value of the mechanical property by exploiting a correlation between the cumulated heat released and the mechanical property. As further explained in the examples that follow, this correlation can be represented using a mathematical relation, which, in the case of certain cements and certain mineral additives, can be a linear relationship.

In addition to the prediction of mechanical properties based on user input, the tool provides a variety of other functionality to aid the design and development of cementitious mixtures. In some embodiments, the tool receives user input corresponding to a desired value of the mechanical property of a cementitious mixture. The user input also can correspond to an age at which the cementitious mixture has the desired value of the mechanical property. Based on the user input, the tool performs calculations using the prediction model to identify a candidate cementitious mixture having a predicted value of the mechanical property that matches the desired value of the mechanical property. Multiple candidate cementitious mixtures can be identified, by iterating through one or more of (a) a size characteristic of the cement, (b) a size characteristic of the mineral addition, and (c) a replacement level of the cement by the mineral addition, as search variables or inputs of the prediction model. For example, by iterating through a size characteristic of the cement and performing calculations to derive predicted values of the mechanical property, the tool can identify one or more candidate size characteristics of the cement that yield matching values of the mechanical property. As another example, by iterating through a size characteristic of the mineral addition and performing calculations to derive predicted values of the mechanical property, the tool can identify one or more candidate size characteristics of the mineral addition that yield matching values of the mechanical property. As a further example, by iterating through a cement replacement level and performing calculations to derive predicted values of the mechanical property, the tool can identify one or more candidate cement replacement levels that yield matching values of the mechanical property.

Alternatively, or in combination with iteratively performing calculations, the tool can identify a candidate cementitious mixture by performing a search through a dataset that is pre-derived using the prediction model, with various combinations of (a) a size characteristic of the cement, (b) a size characteristic of the mineral addition, and (c) a replacement level of the cement by the mineral addition. The dataset also can include experimentally-derived information, computer simulation-derived information, or both.

Matching of a predicted value and a desired value of the mechanical property need not be (but can be) perfect, and the extent of matching of the values can be user-specified, or can be pre-defined, pre-selected, or recommended by the tool, such as to within ±30%, ±25%, ±20%, ±15%, ±10%, or ±5% of the desired value of the mechanical property. In the case that multiple candidate cementitious mixtures are identified, the tool can visually present the multiple candidate cementitious mixtures in a ranked order, based on the extent of matching or other suitable ranking criteria.

The functionality of the tool of some embodiments is further explained with reference to the following examples of user scenarios:

(1). The tool can use a mechanical property or other material property (e.g., compressive strength at 28 days of reaction) as an input in addition to other characteristics of a cement and limestone (e.g., particle size distribution) and can perform calculations to provide suitable mixture proportioning to yield the desired material property.

(2). The tool can recommend alternate mixtures of a cement and limestone which would yield the same or a similar material property. For example, the tool can recommend a high level of coarse limestone replacement that would yield the same or a similar material property as a low level of fine limestone replacement. As another example, the tool can recommend combinations of coarse and fine limestone that would yield the same or a similar material property as the use of fine limestone replacement alone, and can recommend combinations of coarse and fine limestone that would yield the same or a similar material property as the use of coarse limestone replacement alone.

(3). For a given cement replacement level by limestone (e.g., 10% by weight), the tool can provide a recommendation regarding a specific surface area of limestone to yield a desired material property. This information can be used by construction technologists to tailor an average particle size or the surface area of limestone accordingly.

(4). The tool can use a material property as an input and can perform calculations to provide a recommendation on suitable mixture proportioning and a specific surface area of limestone to yield the desired material property.

### EXAMPLES

The following examples describe specific aspects of some embodiments of this disclosure to illustrate and provide a description for those of ordinary skill in the art. The examples should not be construed as limiting this disclosure, as the examples merely provide specific methodology useful in understanding and practicing some embodiments of this disclosure.

### Example 1

### The Filler Effect

### The Influence of Filler Content and Surface Area on Cementitious Reaction Rates

Finely ground mineral powders can be used to accelerate cement hydration rates. This “filler effect” has been attributed to the effects of dilution (an increase in water-to-cement weight ratio w/c) when the cement content is reduced or to the provision of additional surface area by fine powders. The latter contribution (surface area increase) is proposed to provide additional sites for the nucleation of the hydration products, which accelerate reactions. Through experimentation and simulation, this example describes the influence of surface area and mineral type (quartz or limestone) on cement reaction rates. Simulations using a boundary nucleation and growth (BNG) model and a multiphase reaction ensemble (MRE) model indicate that the extent of the acceleration is linked to the: (1) magnitude of surface area increase and (2a) capacity of the filler's surface to offer favorable nucleation sites for hydration products. Other simulations using a kinetic cellular automaton model (HydratiCA) indicate that accelerations are linked to: (2b) the interfacial properties of the filler that alters (increases or decreases) its tendency to serve as a nucleant, and (3) the chemical composition of the filler and the tendency for its dissociated ions to participate in exchange reactions with the calcium silicate hydrate product. The simulations are correlated with accelerations observed using isothermal calorimetry when fillers partially replace cement. This example correlates and unifies the fundamental parameters that drive the filler effect and provides a mechanistic understanding of the influence of fillers on cementitious reaction rates.

As set forth in this example, both experiments and a combination of simulation methods are used to deconvolute the effects of the filler content (cement replacement level and w/c increase) and surface area (fineness) on hydration rates. Computer simulations are applied to describe how a change in the nature and area of the solid surfaces influences reactions. The mechanism of reaction acceleration is investigated for two fillers, namely limestone and quartz, at early ages. The outcomes provide a mechanism for concrete technologists to develop cementitious binders and concretes with reduced clinker factors (for cement) and reduced cement contents (for concretes), which could display similar properties as traditional Portland cements.

### Materials and Experimental Methods

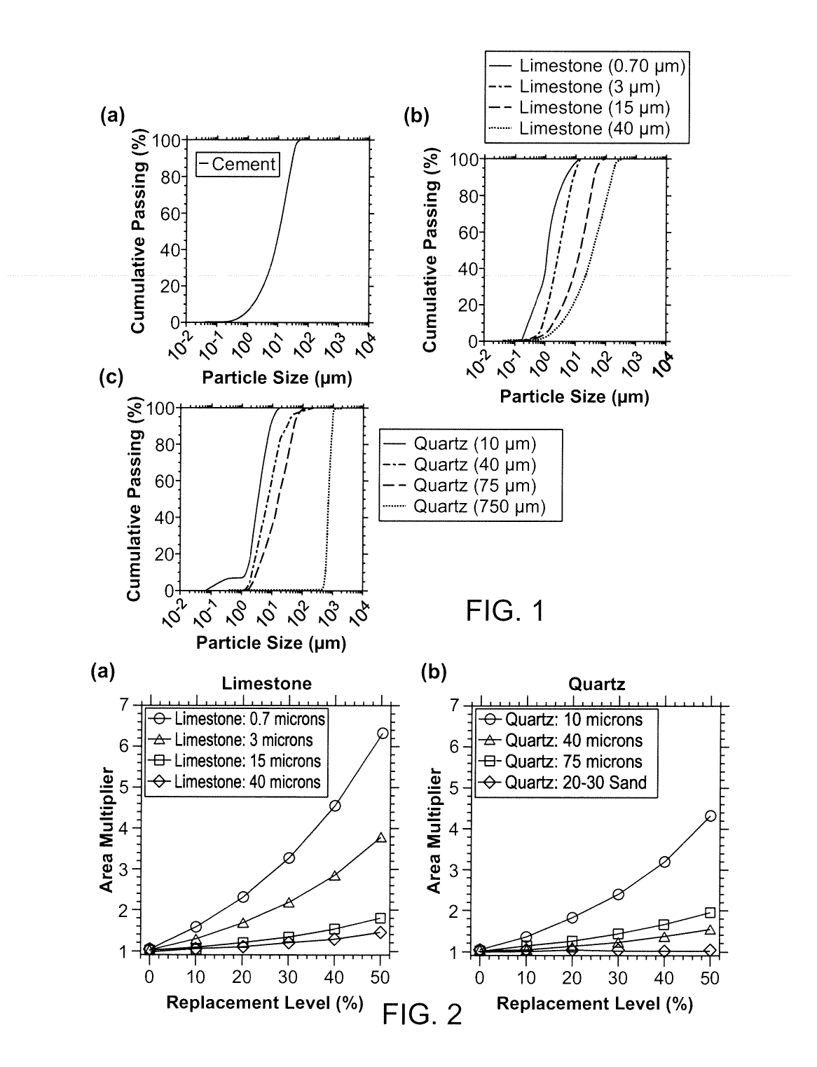

An ASTM C150 compliant Type I/II ordinary Portland cement with an estimated Bogue phase composition of about 59% C₃S, about 16% C₂S, about 4% C₃A, about 11% C₄AF, and a Na₂O equivalent of about 0.40% was used in this example. The limestone and quartz powders used are commercially available (nominally pure) particle size classified products produced by OMYA A.G. (Oftringen, Switzerland) and the U.S. Silica Company (Frederick, Md.). The particle size distributions (PSD, FIG. 1) of all the solids were measured using a Beckman Coulter light-scattering analyzer (LS13-320; Beckman Coulter, Brea, Calif.) using isopropanol and sonication for dispersing the powders to primary particles. The uncertainty in the light-scattering analysis was determined to be about 6% based on multiple measurements performed on six replicate samples assuming the density of the cement, limestone, and quartz to be about 3150, about 2700, and about 2650 kg/m³, respectively.

Cementitious paste mixtures were prepared using deionized water at a fixed water-to-solids weight ratio (w/s=0.45) using a planetary mixer as described in ASTM C305. To better understand the role of fillers, the cement content was progressively reduced (by replacement) in 10% increments from 0% to 50% (mass or weight basis) by limestone and quartz powders of varying particle sizes (FIG. 1 and Table I).

TABLE I
Nominal d₅₀ and Specific Surface Area (SSA) Values, as Calculated Using the
Measured Particle Size Distribution, for the Cement, Quartz, and Limestone Used in this
Example. The Uncertainty in the Measured d₅₀ and SSA are Both About 6%

| Cement | | | Limestone | | | Quartz | | |
|---|---|---|---|---|---|---|---|---|
| | Size (d₅₀) (μm) | SSA (m²/kg) | ID | Size (d₅₀) (μm) | SSA (m²/kg) | ID | Size (d₅₀) (μm) | SSA (m²/kg) |
| Cement | 10.78 | 486.60 | 0.7 | 1.40 | 2592.10 | 10.0 | 3.81 | 1610.00 |
| | | | 3.0 | 2.98 | 1353.20 | 40.0 | 7.42 | 464.50 |
| | | | 15.0 | 14.87 | 399.20 | 75.0 | 17.24 | 270.20 |
| | | | 40.0 | 40.10 | 228.60 | 20-30 Sand | 783.00 | 2.80 |

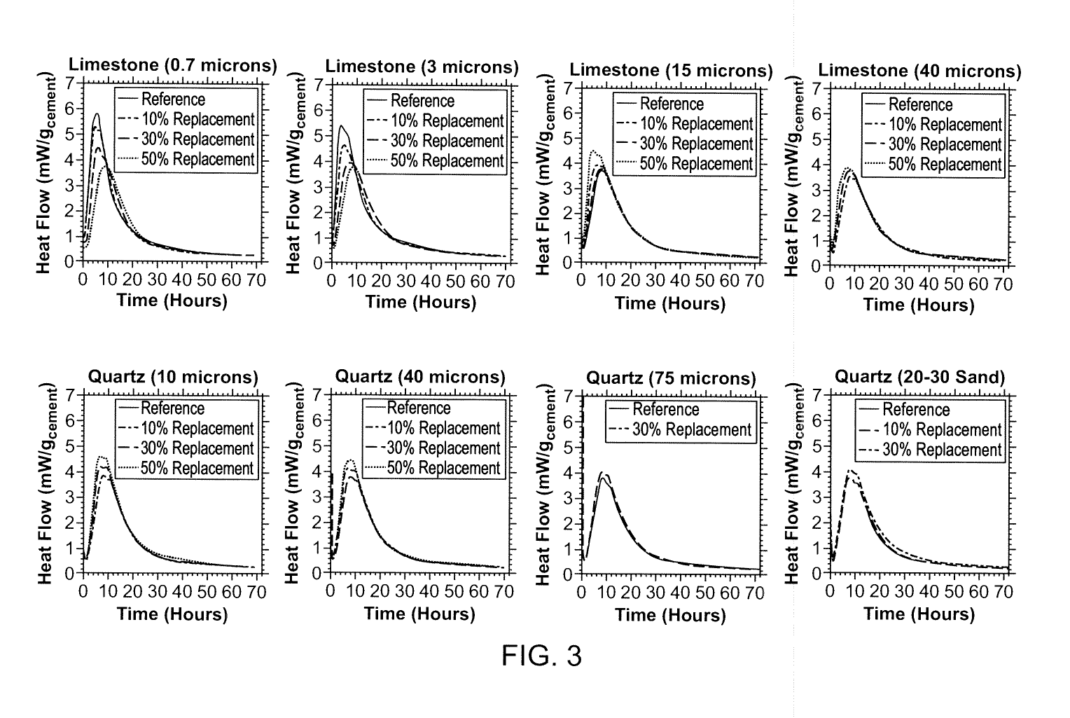

The influence of powder additions (cement replacement) on the solid surface area of the system is shown in FIG. 2 and is described using an area multiplier (AM, unitless) as shown in Eq. (1):

$$AM = 1 + \frac{{rSSA}_{filler}}{( 100 - r ) {SSA}_{cement}} \qquad ( 1 )$$

where r (weight %) is the percentage replacement of cement by filler (limestone or quartz) and SSA_{cement} and SSA_{filler} (m²/g) are the specific surface areas of the cement and filler, respectively, calculated from the particle size distribution and the particle density, while assuming spherical particles. It should be noted that, given the irregular, angular nature of the particles considered, the spherical particle assumption may result in an underestimation of the surface area by a factor of about 1.6-1.8 for typical cement powders. Thus, AM is a scaling factor that describes the (relative) change in solid surface area induced by filler addition in comparison to the surface area provided by a unit mass (1 g) of cement. In other words, AM is the surface area of filler per unit surface area of cement in the system. The greater this quantity is, either because the filler is finer or because it is present in greater amounts, the more AM will exceed unity. It should be noted that the calculation of AM can be subject to uncertainties that stem from measurements of the PSD.

The influence of cement replacement on the rate of reactions was tracked using isothermal conduction calorimetry. A TamAir isothermal calorimeter (TA Instruments, New Castle, Del.) was used to determine the heat evolved during hydration, of externally mixed pastes, at a constant temperature condition of 25° C. The thermal power and energy measured were then used to assess the influence of powder additions on reaction kinetics and cumulative heat release of the cementitious pastes. The uncertainty in the measured heat flow rate was determined to be about ±2% based on the heat flow measured on six replicate specimens between 1 and 72 h.

### Experimental Results

### Assessing the Heat Release Response using Isothermal Calorimetry

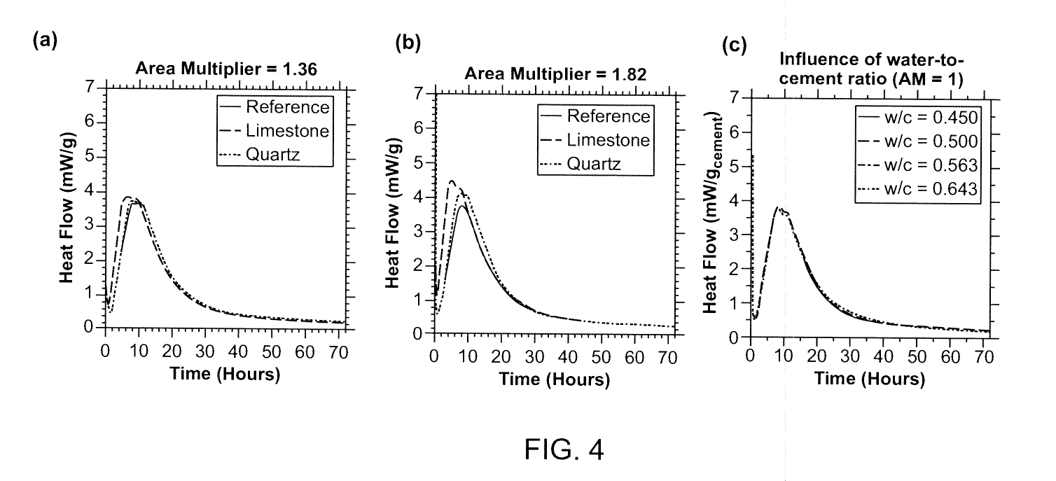

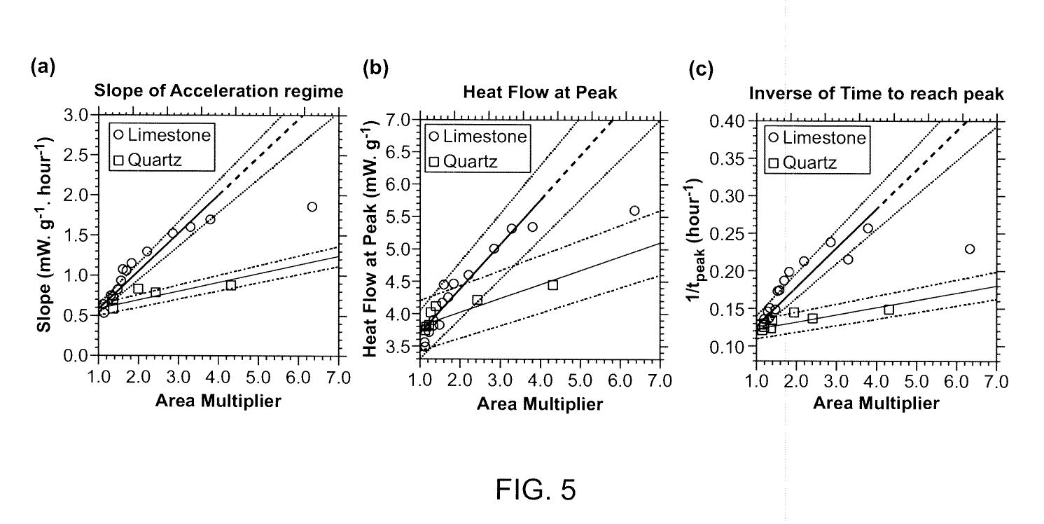

FIG. 3 shows representative heat evolution profiles for plain and binary (cement and limestone or cement and quartz) pastes for different levels of cement replacement. As denoted by the left shift of the rate curve, the rate of reactions increases with the cement replacement level and filler fineness. It is noted that even for similar or identical contributions of solid surface area, limestone is a better accelerant of hydration reactions than quartz (FIG. 4).

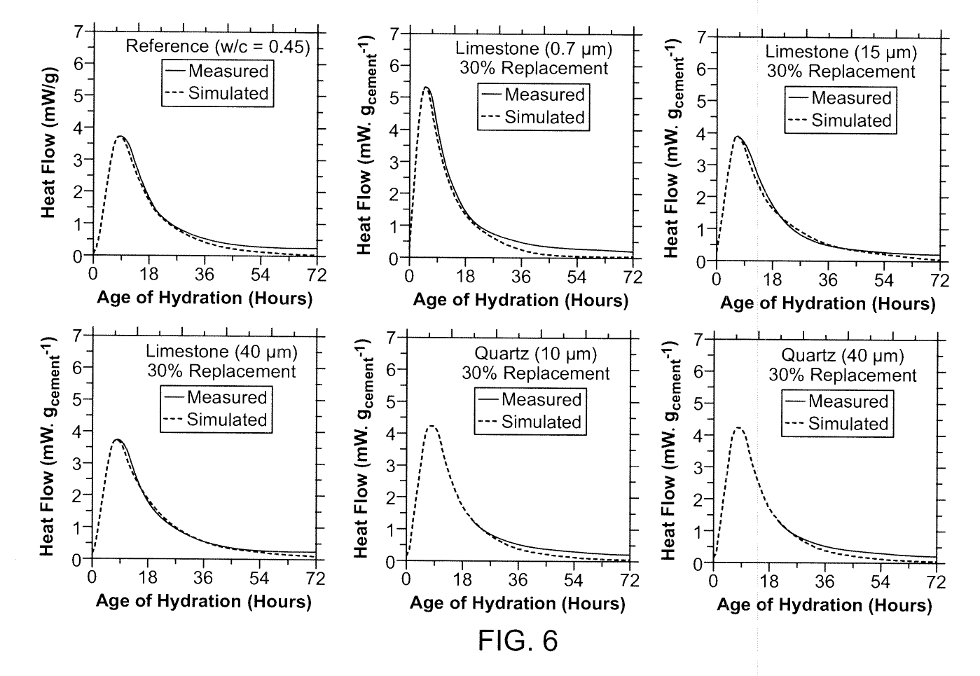

Given the large quantity of data produced, to describe the heat release responses of the mixtures, and their differences with respect to the (pure cement paste) reference more quantitatively, the heat curves were parameterized to determine the: (a) slope during the acceleration regime, (b) inverse of time elapsed from initial water contact to the main heat peak, and (c) amplitude of the heat peak (the heat flow at the peak) for each mixture. FIG. 5 indicates that the rates of reactions are enhanced in proportion with AM; both the slope during acceleration (FIG. 5(a)) and the maximum heat flow rate increase (FIG. 5(b)). As can be appreciated, this acceleration corresponds to a reduction in the time to reach the peak (FIG. 5(c)). Furthermore, note that all points, but one, are within about 10% bounds of the best-fit trend lines; the lone exception corresponds to a high AM value (0.7 μm limestone, 50% replacement), which shows less than expected acceleration. This deviation may result from either or both: (1) enhanced agglomeration of fine filler particles, which would effectively act to reduce their exposed surface area and would trap water inducing a less than expected acceleration in hydration rates and (2) a surface area saturation effect, wherein for AM>4, the available surface area is more than is required for reaction of the available quantity of cement, resulting in a plateau in the measured reaction parameters. The examples below detail analytical methods by which reaction parameters, such as those illustrated in FIG. 5, can be related to property (compressive strength) development in cementitious materials.

To compare their relative influences, it should be noted from FIG. 5 that both limestone and quartz accelerate hydration reactions in terms of reducing the time to the heat peak and increasing the peak height at equal AMs. But the effect is much more pronounced for limestone than for quartz according to both of these measures, as also noted in FIG. 4.

To further quantify the heat release response and deconvolute the effects of dilution and of increased surface area, a set of plain cement pastes were prepared with w/c ratios corresponding to the actual cement content in the systems with partial filler replacement levels ranging from 0% to 30% by weight (FIG. 4(c)). In spite of a changing w/c, (as AM=1 for all systems), the heat flow rates normalized by weight of cement are essentially identical. This result indicates that the reaction kinetics are largely independent of water content unless additional surface area is provided by fillers. This result may indicate that, for the range of plain pastes and the w/c evaluated, the amount of water available to the reactant particles (the water-to-cement distance function) in realistic systems is broadly similar, and is mainly a function of a similar level of solid agglomeration in these systems. It is expected that there is a lower limit of w/c (e.g., w/c<0.42) below which cement hydration rates begin to be influenced by the growing scarcity of water, especially at later ages as hydration progresses and self-desiccation occurs

Computational Simulations of the Heat Release Response:

To more rigorously interpret the calorimetric parameters, the heat release response was simulated using three models: (1) BNG, (2) MRE, and (3) kinetic cellular automaton model (HydratiCA). The simulations are applied to develop a mechanistic, physically consistent basis for understanding the influence of fillers on hydration reaction rates. It should be noted that the BNG and MRE models are applied to simulate the postinduction period of hydration, and that their results presented here are the best fits obtained for the corresponding (measured) systems. A best fit is described as a simulation result that falls within a 5% bound of the measured heat evolution profile for more than 90% of the time between 2 and 72 h. A sequence based on the simplex method is utilized to optimize the simulation parameters for a given system. The optimization procedure involves: (a) providing w/c, SSA_{cement}, and the measured heat flow as inputs, (b) defining different simulation parameters as either variable or fixed (see summary below for fixed and variable parameters), and (c) defining constraints, or numerical bounds, on the variable simulation parameters. Initial guesses for fixed and variable parameters are the ones used for the paste system with no filler. The simplex method is invoked to iterate the values of the variable parameters within predefined constraints until the error between the measured and calculated rate curves is minimized between 2 and 72 h. Through the iterations, the step size of each variable parameter is set at 0.0005 units and the numerical tolerance set to 10⁻¹⁴. The optimization sequence is deemed to have converged when the magnitude of the difference in errors from two consecutive iterations is less than the set numerical tolerance. This convergence criterion mitigates against the potential for numerical oscillations in the solution and yields the optimum values of the variable simulation parameters for a given system.

(1) Classical Boundary Nucleation and Growth

Classical and modified forms of BNG models can be applied to describe the hydration of cementitious systems. These models simulate reactions as a nucleation and growth process that starts at solid-phase boundaries. In these models, a single product of a constant density is assumed to form, and its nucleation or growth is treated as the rate-controlling mechanism that determines the kinetics of the reaction. BNG models have been formulated with a variety of assumptions for reaction mechanisms, including nucleation site saturation, product growth control, and the continued nucleation of product phases. This example applies a modified form of a BNG formulation as shown in Eqs. (2-6):

$$X = 1 - exp [ - 2 {a}_{BV} \text{?} ( 1 - exp ( - \text{?} ) ) d y ] \\ \text{?} \text{indicates text missing or illegible when filed} \qquad ( 2 )$$

where X is the volume fraction of the reactant transformed into product, Gₒᵤₜ is the outward growth rate of the product, a_{BV} is the boundary area per unit volume, y is an integration variable, t is the simulation time (h), and A_{f} is the extended area (dimensionless) of the transformed product described in Eqs. (3) and (4):

$$\begin{matrix} {A}_{f} = π [ \begin{matrix}{I}_{density} \cdot {G}_{par}^{2} \cdot ( {t}_{r}^{2} - \frac{{y}^{2}}{{G}_{out}^{2}} ) + \\ {I}_{rate} \cdot {G}_{par}^{2} \cdot ( \frac{{t}_{r}^{3}}{3} - \frac{{y}^{2} {t}_{r}}{{G}_{out}^{2}} + \frac{2 {y}^{3}}{3 {G}_{out}^{3}} )\end{matrix} ] \\ if ( \text{?} > \frac{\text{?}}{{G}_{out}} ) & ( 3 a ) \\ \text{?} = π [ \begin{matrix}{N}_{density} \cdot ( \text{?} - \frac{{y}^{2}}{{G}_{out}^{2}} ) + \\ {N}_{rate} \cdot ( \frac{{t}_{r}^{3}}{3} - \frac{{y}^{2} \text{?}}{{G}_{out}^{2}} + \frac{2 {y}^{3}}{3 {G}_{out}^{3}} )\end{matrix} ] \\ if ( \text{?} > \frac{\text{?}}{{G}_{out}} ) & ( 3 b ) \\ {A}_{f} = 0 if ( \text{?} \leq \frac{y}{{G}_{out}} ) \\ where , & ( 4 a ) \\ ( {t}_{r} = ( t - {t}_{0} ) ) \\ \text{?} \text{indicates text missing or illegible when filed} & ( 4 b )\end{matrix}$$

where I_{density} (μ⁻²) is the nucleation density of the product, that is, the starting number of supercritical nuclei per unit surface area, Iᵣₐₜₑ (μm⁻²/h) is the nucleation rate, Gₚₐᵣ (μm/h) is the growth rate parallel to the boundary surface, and Gₒᵤₜ (μm/h) is the outward growth rate, perpendicular to the particle surface. Eq. (3) can also be expressed using Nᵣₐₜₑ (h⁻³) and N_{density} (h⁻²), as shown in Eq. (3a), which are, respectively, the products of the nucleation rate and nucleation density with the square of the parallel growth rate (Iᵣₐₜₑ−Gₚₐᵣ² and I_{density}·Gₚₐᵣ²), shown in Eq. (3b). The latter form, where the nucleation rate and nucleation density are convoluted with the parallel growth rate, is a more accurate representation of systems with anisotropic growth of product because in these systems the fraction of area covered at a given distance from the nucleation (and growth) boundary depends on contributions from existing nuclei (present at a given time) and their growth rate along the boundary. Therefore, for a given N_{density} or Nᵣₐₜₑ, different combinations of I_{density}, Iᵣₐₜₑ, and Gₚₐᵣ may be permissible. The rate of heat release due to the hydration of the reactant (alite or cement) is computed using a scaling parameter, A (kJ/mol), as shown in Eq. (5):

$$( \frac{d H}{d t} ) = A \cdot ( \frac{100}{100 - r} ) \frac{d X}{d t} \qquad ( 5 )$$

where r (%) is the (weight) percentage replacement level of filler which accounts for the effects of dilution (a reduction in reactive cement content). In addition, the simulation begins at the end of the induction period, so the simulation time is mapped to real time by using a parameter t₀ to designate the time at which the induction period ends as described by Eq. (4b). The boundary area per unit volume, a_{BV} (μ⁻¹), is calculated by adding the surface areas of the cement and filler and dividing by the system volume (total solids plus water):

$${a}_{BV} = \frac{{SSA}_{cement} \cdot {a}_{factor} \cdot {ρ}_{cement} \cdot ( 100 \cdot {f}_{cement} )}{{V}_{free}} \qquad ( 6 )$$

where f_{cement} (unitless) is the initial volume fraction of cement, ρ_{cement} is the density of the cement (3.15 g/cm³), V_{free} (μm³) is initial volume of water present in the system, and SSA_{cement} is the specific surface area of cement fixed at 486.00 m²/kg. The parameter a_{factor} (unitless) acts as a free variable representing a “virtual AM” used in the simulations. Based on the optimum parameters obtained for simulations of Portland cement systems, for all simulations, the values of Iᵣₐₜₑ, Gₒᵤₜ, and Gₚₐᵣ are fixed at 0.0 μm⁻²/h, 0.03 μm/h, and 4.0 μm/h, respectively—indicative of a site saturation assumption. Next, f_{cement} (unitless) and a_{BV} (μm⁻¹) serve as input variables, whereas A (kJ/mol), I_{density} (μm⁻²), a_{factor} (unitless), and t₀ (h) remain free (fitting) variables. Selection of a different value of Gₚₐᵣ would lower or enhance the values of I_{density} proportionally, but would not otherwise alter the outcomes, or trends, identified by the simulations.

First, the best-fit values of the simulation variables for the plain paste system were identified as rough estimates from prior work conducted on plain paste systems with similar surface areas and compositions and fine-tuned to properly describe the current paste system. Second, to fit the binary pastes with different levels of filler replacement, the simplex method described earlier was applied to find the optimum parameters by varying: (1) I_{density} and a_{factor} from the values determined for the reference system to match the upslope and the time of peak through the acceleration regime, (2) the parameter A to be scaled so as to match the amplitude of the heat flow rate at the main peak, and (3) t₀ to shift the simulated heat flow response to the right (increase t₀) or to the left (decrease t₀) to temporally match the measured heat response.

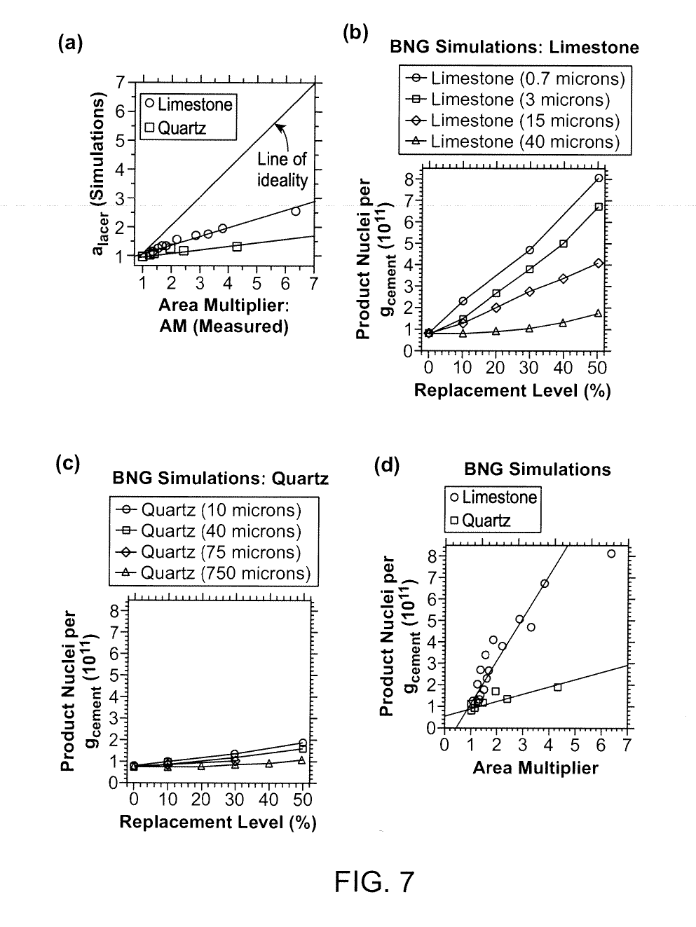

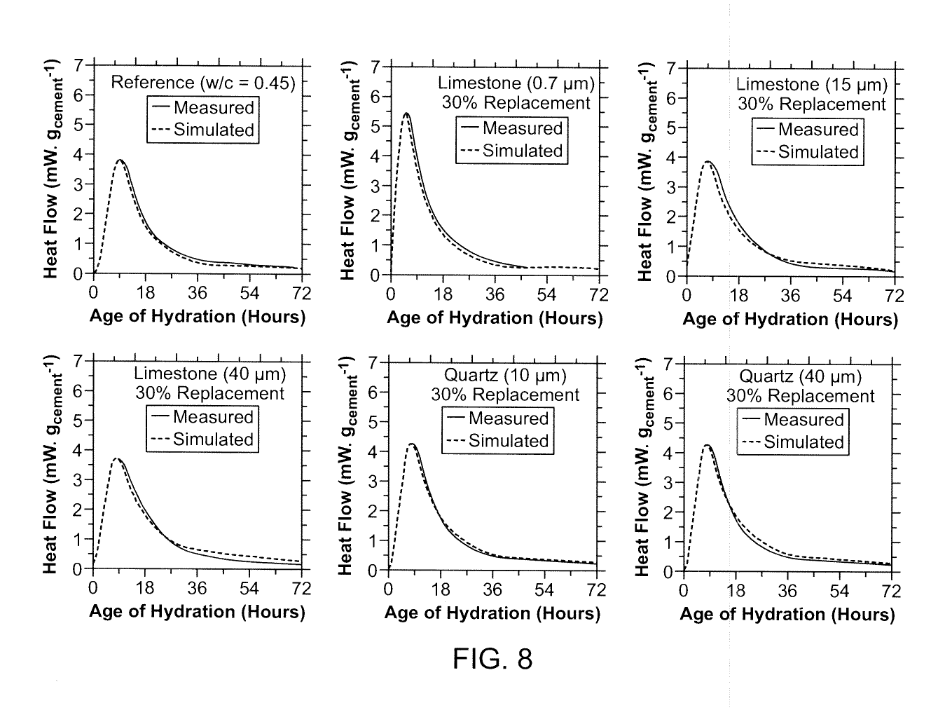

FIG. 6 shows representative best-fit simulation results for the reference and binary paste systems. Good fits are obtained for the reference system and for systems having low and intermediate levels of cement replacement. Although the quality of the fit does slightly degrade at higher levels of cement replacement (approaching about 50%, weight basis), the BNG approach is broadly able to simulate the measured heat response. The parameter optimizations suggest that A decreases with increasing replacement levels, although no systematic trend could be found in its variation with respect to filler content, type, or surface area. The values of a_{factor} (virtual AM) are consistently less than the actual AM (FIG. 7(a)) for both limestone and quartz systems, with a_{factor} varying about linearly with AM, and with slopes significantly less than unity. Nevertheless, a_{factor} is much more sensitive to limestone replacement than to quartz replacement. This trend indicates that a fraction of the filler's total surface area can offer preferential nucleation sites for the reaction products. However, a larger fraction or equal fraction at higher efficiency of the limestone surface participates in reactions compared with quartz. This aspect begins to explain how fine limestone is a more capable mineral acceleration agent than quartz, a point which is discussed in more detail below.

Next, the fitting parameters a_{factor} and I_{density} are combined to calculate the number of supercritical product nuclei, N_{nuc}, produced per gram of reactant as shown in Eq. (7):

N_{nuc}=(SSA_{cement}a_{factor})I_{density} (7)

The number of supercritical product nuclei produced per gram of cement is plotted against the level of cement replacement (FIGS. 7 (b) and (c)) and AM (FIG. 7(d)). As can be appreciated, increasing cement replacement results in a proportional increase in the number of nuclei that participate in chemical reactions. This trend indicates increased product nucleation (higher I_{density} values, while Iᵣₐₜₑ remains fixed) and therefore greater reaction rates in the presence of either mineral filler as compared with the plain cement system. However, limestone displays a substantially amplified nucleation response compared with quartz because, both at equal replacement levels (FIGS. 7(b) and (c)) and equal AM values (FIG. 7(d)), a larger number of product nuclei are initially generated in systems containing limestone. The divergence of the quartz and limestone response noted in FIG. 7(d) correlates well with experiments (FIG. 5). It is reasonable to expect that the number of nuclei would elevate with an increase in the surface area, but this response is filler specific. The divergence in the limestone and quartz responses is then indicative of the differing ability of these two minerals to serve as hydrate nucleation surfaces and mineral acceleration agents, with limestone showing a far greater surface affinity for the nucleation and growth of the cement hydrates.

(2) Multiphase Reaction Ensemble

The MRE is a thermokinetic hydration model that uses inputs of the phase composition and particle size characteristics in conjunction with thermokinetic rules to simulate hydration. The model omits contributions from the belite and ferrite phases in the first 3 d of hydration. First, to simulate alite hydration, the model applies a nucleation and densifying growth criteria in which the C—S—H is assumed to grow with an increasing density with time. Briefly, the incremental amount of alite consumed by hydration in a time step dt is given by:

$$\begin{matrix}- {dm}_{alite} = \frac{1}{k} [ ( \frac{\text{?}}{{ρ}_{0}} ( d {V}_{extended , CSH} ( t + d t ) - d {V}_{extended , CSH} ( t ) ) ( 1 - {V}_{real , solid} ) ) + ( \frac{ρ ( \text{?} + d t ) - ρ ( \text{?} )}{{ρ}_{0}} {V}_{real , CSH} ) ] & ( 8 a ) \\ Here \\ \frac{d {V}_{real , CSH}}{d t} = \frac{d {V}_{extended , CSH}}{d t} ( 1 - {V}_{real , solid} ) & ( 8 b ) \\ {V}_{real , solid} = \frac{\text{?} - \text{?}}{\text{?}} \\ \text{?} \text{indicates text missing or illegible when filed} & ( 8 c )\end{matrix}$$

The first term in Eq. (8a) describes the amount of C—S—H formed, the second term describes the incremental change in volume of C—S—H that already exists (that was formed between time t, and t), the parameter k is the ratio of the mass of alite reacted to the mass of C—S—H produced, t is the simulation time, and ρ₀ (g/cm³) is the base density of C—S—H fixed at 2.10 g/cm³. V_{real,C—S—H} (volume fraction) and V_{extended,C—S—H} (volume fraction) are the volume fractions of the phase whose growth controls the kinetics, C—S—H in this case, without and with consideration of overlaps in surfaces, respectively, and V_{real,solid} (volume fraction) is the fractional increase in the total volume of solids (V_{solid}) in the representative elementary volume (REV=100 μm³) at time t. These equations account for the space occupied by each phase (unreacted alite, portlandite, and C—S—H) and the progressive change in the volume of C—S—H that is already present and continues to form with increasing hydration. The extended volume of the hydration product at any time can be calculated according to:

V_{extended,CSH}=∫₀^{G}^{_{out,t}}a_{BV}(1−exp(−A_{f}))dy (9)

The C—S—H density is assumed to vary exponentially in time according to:

$$ρ ( t ) = {ρ}_{max} - ( {ρ}_{max} - {ρ}_{min} ) \cdot exp [ \frac{- {k}_{den} \cdot ( t - {t}_{0} )}{( {ρ}_{max} - {ρ}_{min} )} ] \qquad ( 10 )$$

where t₀ (h) is the start time parameter and ρₘₐₓ (2.10 g/cm³) is the final density of the outer C—S—H, ρₘᵢₙ (g/cm³) is the initial density of outer C—S—H, k_{den} (g/cm³ per hour) is the rate of densification of outer C—S—H, a_{BV} is the boundary area of alite per unit volume (μm⁻¹) calculated using Eq. (6) with SSAₐₗᵢₜₑ=fₐₗᵢₜₑ·SSA_{cement}. Here, fₐₗᵢₜₑ is the mass fraction of alite in the cement determined using quantitative X-ray diffraction. For these simulations, the values of Iᵣₐₜₑ, Gₒᵤₜ, k_{den}, and Gₚₐᵣ are fixed at 0.05 μm⁻²/h, 0.1035 μm/h, 0.00055 g/cm³/h, and 1.0 μm/h, respectively. The free variables for the alite hydration sequence are ρₘᵢₙ (g/cm³), I_{density} (μm⁻²), a_{factor} (ratio), and t₀ (h).

Second, aluminate reactions were simulated in two stages. Stage 1 describes C₃A hydration in a sulfated solution, which results in ettringite precipitation, and is modeled by a first-order rate law:

$$\begin{matrix} \frac{d {m}_{{C}_{3} A}}{d t} = - \text{?} - {a}_{SA} \\ where , & ( 11 ) \\ {a}_{SA} ( \text{?} ) = \text{?} \cdot 4 {π ( ( \text{?} ) - \text{?} )}^{2} \\ where , & ( 12 ) \\ \text{?} = ( \frac{3 {v}_{cement}}{4 π} ) \text{?} \\ \text{?} \text{indicates text missing or illegible when filed} & ( 13 )\end{matrix}$$

where V_{cement} (cm³) is the volume of cement in the system, f_{C3A} is the C₃A content (weight fraction) of the cement, k₁ is a reaction rate constant (cm/h), t is the time (h), and c_{C3A} (g/h/cm²) is a dimensional matching (normalization) constant. In this model, therefore, the cement is assumed to be assembled into a single (hypothetical) spherical particle, the radius of which decreases with time, and the surface area of C₃A changes in proportion to that of the single particle. The values of k₁ and c_{C3A} were determined to be constants at values of 0.125 (cm/h) and 7.59×10⁻⁷ (g/h/cm²), respectively.

Stage 2 of C₃A hydration covers the period after sulfate depletion, when ettringite does transform into monosulfate, and is modeled by a BNG mechanism, Eqs. (2-5). This choice is based on observations of the hydration of model (mechanical) mixtures of C₃A gypsum systems, in which the heat release after gypsum depletion can be fit by a nucleation and growth equation. For this stage, the values of Gₒᵤₜ, Gₚₐᵣ, Iᵣₐₜₑ, I_{density}, and t₀ are fixed at 0.003 μm/h, 1.0 μm/h, 0.05 μm⁻²/h, 0.0 μm⁻², and 18 h, respectively. The value of a_{BV} is obtained using the value of a_{SA} from Eq. (12) at t=t₀, which represents the start time for monosulfate formation. The value of t₀ is fixed at 18 h for all systems considered, which corresponds to the time of gypsum depletion in the reference system, as determined from modeling of Stage 1.

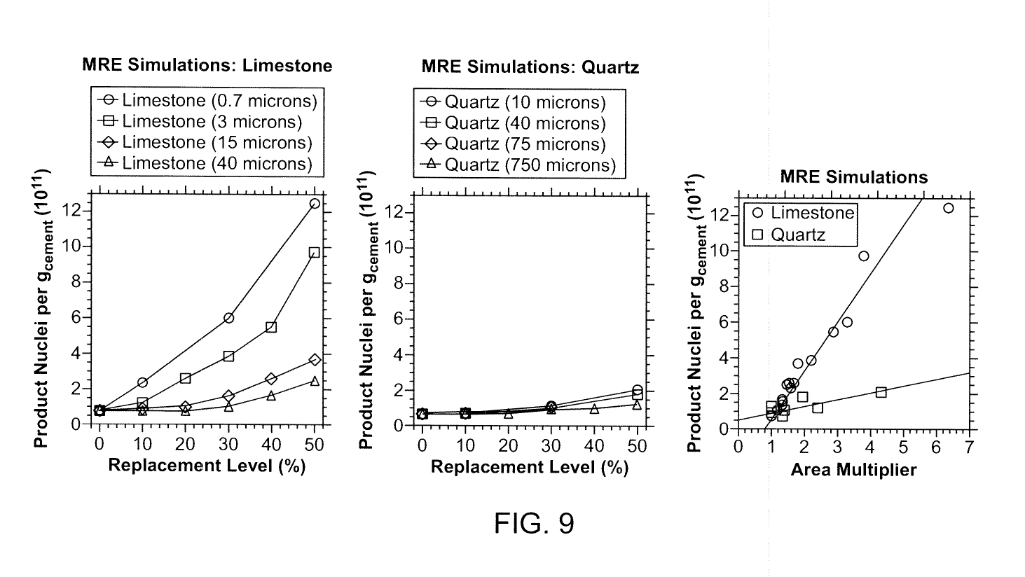

Using the MRE model described, alite hydration and aluminate hydration are assumed to be chemically decoupled, and therefore are treated separately so that the heat evolved from their respective reactions is added to obtain the heat profiles shown in FIG. 8. Here, the best-fit values of the simulation variables for the plain system were first identified as estimates and then fine-tuned to properly describe the heat curve of the reference (plain paste) system. For binary paste systems, once again, the simplex method described previously was used, with I_{density} and a_{factor} being varied from their values in the reference system to best match the upslope and the time of peak during the acceleration regime. In addition, ρₘᵢₙ and t₀ were also varied to match the amplitude of the heat flow at the main peak (analogously to the parameter A) and to shift the simulated heat flow to the right (increase t₀) or left (decrease t₀) along the x-axis.

FIG. 8 shows representative best-fit simulation results for the reference and binary paste systems using the MRE model. FIG. 8 shows that the MRE simulations are able to reliably replicate the experimental results for the entire range of systems and all cement replacement levels. However, relatively large variations in a_{factor} (as relevant to the filler content and fineness) and I_{density}, and relatively smaller variations in t₀ (−1.20 to −2.10 h) and ρₘᵢₙ (0.196 to 0.390 g/cm) were implemented to obtain good fits. It should be noted that variations in t₀ are applied to account for changes in the duration of the induction period (start time of the acceleration regime) because systems containing fillers often experience a slightly shorter induction period than the reference paste system. Variations in ρₘᵢₙ (increasing ρₘᵢₙ with replacement level and filler fineness) are implemented to scale the amplitude of the simulated heat flow.

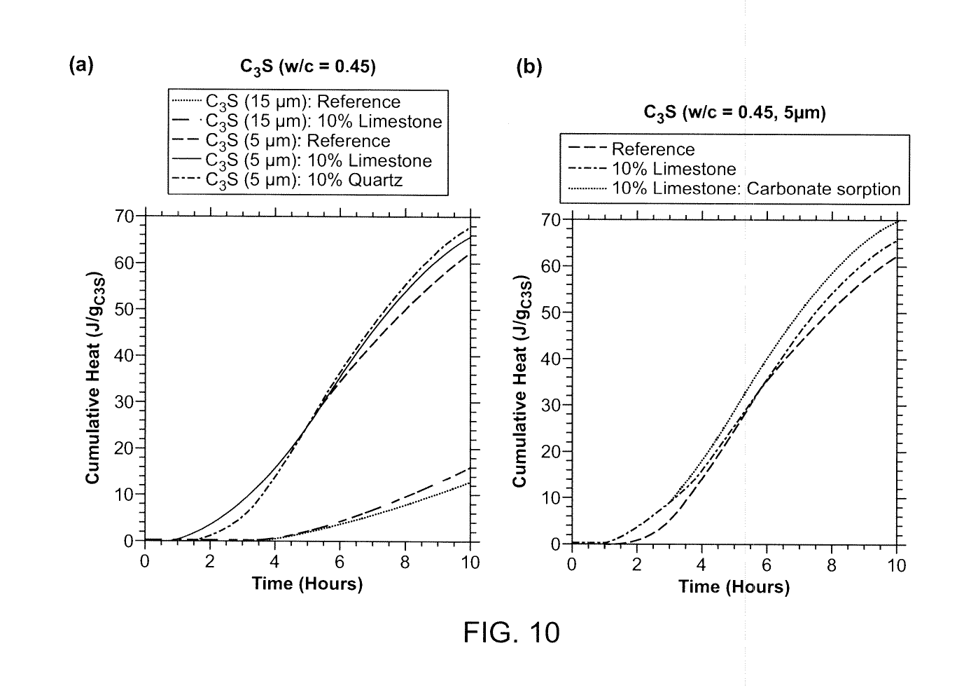

As in the analysis of the BNG simulations, the nucleation density (I_{density}) and area factor (a_{factor}) are combined to calculate the number of supercritical product nuclei associated with a specific system. FIG. 9 shows the number of nuclei as a function of the cement replacement level (FIGS. 9 (a) and (b)) and as a function of AM (FIG. 9(c)) for systems with limestone or quartz. Once again, increasing replacement of cement and solid surface area both increases the number of supercritical nuclei participating in the reactions. The divergence noted in the limestone and quartz systems (FIG. 9(c)) is consistent with trends identified in the measured calorimetric parameters (FIG. 5). Therefore, the MRE results, in agreement with the BNG simulations, indicate that: (1) the additional surface area provided by fillers can enhance the nucleation of the hydration products and hence the rate and extent of early-age hydration reactions, and (2) quartz and limestone can both enhance reaction rates, but limestone has a greater accelerating capacity than quartz at a given AM, due to its higher nucleation potential (number of supercritical nuclei produced and trends in I_{density}).

(3) Kinetic Cellular Automata Simulations (HydratiCA)

Cellular automata models can be used to simulate chemical and structural changes in space and time within systems by discretizing space and matter into uniform lattice sites and concentration quanta, respectively. A kinetic cellular automata model (HydratiCA) for simulating diffusion, advection, and homogeneous rate kinetics in reactors has been adapted to simulate chemical and structural evolution during early-age hydration of cement. This model is applied to investigate how the thermodynamics and kinetics of C—S—H nucleation on surfaces of C₃S, limestone, and quartz can influence hydration and microstructure evolution at early ages. Chemical changes and microstructural development are simulated by iterating over small time steps Δt, typically about 0.1 ms. Time steps are split into a transport step, during which mobile components in solution are able to move between lattice sites according to diffusion (random walk) or by perfect mixing (instant homogenization, as implemented in this example), and a reaction step, during which reactant species may combine to form products according to defined stoichiometric reaction equations. The probability, pᵢ, of reaction i occurring at a lattice site depends on its relative rate constant, kᵢ, and on the number of cells N_{a,i} of each reactant, a, involved in the reaction as shown in Eq. (14):

$$\text{?} = \text{?} \text{?} max ( 0 , \text{?} \text{?} - m + 1 ) \\ \text{?} \text{indicates text missing or illegible when filed} \qquad ( 14 )$$

where ξ is a constant model parameter that relates Nₐ to the molar concentration of component a, and vₐ is the molar stoichiometric coefficient of component a in the reaction. The relative rate constant is the product of the absolute forward rate constant, k_{i,+}, and the linearized thermodynamic driving force,

kᵢ=k_{i,+}(1−Sᵢ) (15)

where Sᵢ, the saturation index for reaction i, is defined as the quotient Kᵢ/K_{i,eq} of its activity product and its equilibrium constant for the forward reaction. For heterogeneous reactions (reactions restricted to a surface), the surface area intersected by the lattice site is multiplied on the right side. Eq. (15) is strictly applicable for elementary reactions (those involving one molecular step), but it can be a useful approximation for many of the more complex dissolution and growth reactions that occur during cement hydration. If kᵢ is negative in Eq. (15), the reaction is eligible to proceed in the reverse direction, in which case products are treated as reactants and vice versa for a given relative rate constant |kᵢ|. The reaction is allowed if pᵢ in Eq. (14) exceeds a random number drawn from a uniform distribution on [0, 1]. When a reaction happens, the number of cells of each reactant (product) at the affected lattice site is decremented (or incremented) by the number indicated by the molar stoichiometric coefficients v.

Eqs. (14) and (15) are sufficient for modeling reaction kinetics involving dissolution, growth, sorption, and ion complexation. As this example is concerned with the kinetics of hydration in the presence or absence of fillers that might offer a reduced barrier for nucleation of C—S—H, to further consider these aspects, nucleation rates are modeled using nucleation theory. The number of supercritical nuclei formed per unit volume per unit time (the nucleation rate) is given by Eq. (16):

I=gSe^{-W*/k}^{_{B}}^{T} (16)

where g (s⁻¹) is the attempt frequency (or “frequency factor”), W* (J) is the work to form one supercritical nucleus, k_{B} is Boltzmann's constant, and T is the absolute temperature (K). W* itself is not a constant, but rather depends on temperature, the surface energy (γ, J/m²) of the nucleating phase in the parent solution, and the saturation index, S, of the solution:

$$\text{?} = \frac{A {Ω}^{2} {γ}^{3}}{\text{?} {ln}^{2} S} = \frac{{k}_{B} \text{?}}{{T}^{2} {ln}^{2} S} \\ \text{?} \text{indicates text missing or illegible when filed} \qquad ( 17 )$$

where A is a geometric factor, Ω is the molecular volume (m⁻³) of the nucleating phase, and w* (J) is approximately constant for a given nucleating material and parent solution. Thus, the rate of Eq. (16) can be mapped to a probability equation similar to Eq. (14), except that in this case the relative rate constant kᵢ is replaced by k_{nuc}:

$$\text{?} = {k}_{0} exp ( \frac{- \text{?}}{\text{?} {ln}^{2} S} ) \\ \text{?} \text{indicates text missing or illegible when filed} \qquad ( 18 )$$

This stochastic model was used to simulate early hydration in a C₃S suspension (w/s=0.45) with or without 10% replacement by weight of quartz or limestone particles. Some of the reactions and their associated thermodynamic and kinetic input parameters are provided below. Because simulations using this model are computationally intensive, and because the objective in using the model is to investigate the influence of C—S—H nucleation parameters on hydration rates, simulations were carried out for small systems containing a single C₃S particle, either 5 or 15 μm in diameter and, in selected simulations, a random dispersion of filler particles in the solution surrounding the C₃S particle. Periodic boundary conditions are invoked to compensate for the finite system volume.

In these simulations, the work of nucleation of either form of C—S—H on C₃S surfaces (w*) is assumed to be 10^{10.83} K³, which is comparable to that for some inorganic salts nucleating in aqueous solutions. On limestone surfaces, the work of nucleation is assumed to be lower than this value by a factor of 4. The attempt frequency is assumed to be 10^{14.2} s⁻¹. In systems with limestone replacement, it is expected that carbonate anions are incorporated within C—S—H, to a certain extent, by analogy to the observed uptake of sulfate ions in systems containing gypsum. It is assumed that this incorporation occurs via the same kind of ion-exchange reaction as that used to model sulfate incorporation in CSH:

CSH(II)+CO₃²⁻→C—\ddot{C}—S—H+2OH⁻, k₊100 mol/m³/s, K_{eq}=10^{3.57} (19)

As a first approximation, solely the CSH(II) form is assumed to participate in the ion-exchange reaction because the sorption tendency of anions should decrease with decreasing Ca/Si ratio as the zeta potential decreases. In the case of limestone dissolution, it is assumed that the limestone used is pure calcite. The forward rate constant is assumed to be k⁺=0.72 μmol/m²/s and the equilibrium constant is K_{eq}=10^{-8.48}, with an enthalpy of reaction of −14.8 kJ/mol (exothermic). A number of ion-ion complexation reactions occur in solution, but two are expected to primarily influence the results:

CaOH⁺→Ca²⁺+OH⁻, k₊=0.06 mol/m³/s, K_{eq}=0.0603 (20)

CO₃²⁻+H₂O→HCO₃⁻+OH⁻, k₊=0.06 mol/m³/s, K_{eq}=10^{-3.67} (21)

The rate constants are chosen to be large enough that the reactions occur very rapidly compared with other dissolution and growth reactions, but otherwise the values are arbitrary. The enthalpy of the former, carbonate reaction is 41 kJ/mol (endothermic). The enthalpy of the other reaction is not calculated from thermodynamic datasets, but it is not expected to make a significant contribution to the heat signature of a hydrating cementitious system.

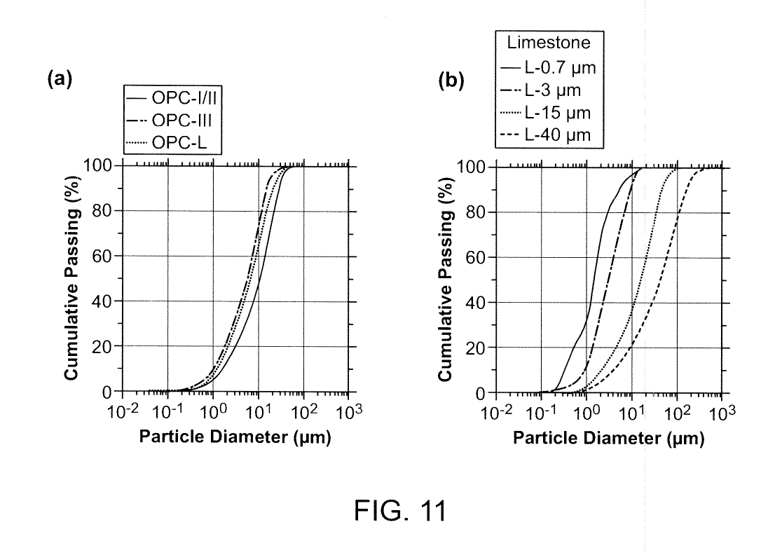

FIG. 10(a) shows the simulated cumulative heat release per gram of reactant for a system with either a 5 μm C₃S particle or a 15 μm C₃S particle with no limestone filler, both of these systems each with 10% weight replacement by limestone filler that offers a lower energy barrier than C₃S for C—S—H nucleation, and a system with the same replacement level for quartz filler where the energy barrier for the nucleation of C—S—H on quartz and on C₃S is equal. The model tracks heat release by multiplying the number of times each unit reaction occurs by the enthalpy change for each reaction. Enthalpies of the dissolution and precipitation reactions for phases, including C₃S, portlandite, C—S—H (I)m and C—S—H (II), and for diffusive transport rates through the C—S—H forms are obtained from published sources.

FIG. 10 (a) shows that limestone causes a shortening of the induction period by as much as about 50% when it provides a lower C—S—H nucleation barrier (“a preferred filler effect”), although the effect is much greater for smaller particles. This behavior is also consistent with the BNG and MRE results already discussed. In contrast, little or no acceleration is predicted during the first 5 h of hydration when nucleation on a filler (in this case quartz) has the same energy barrier as on C₃S, although at later times the cumulative heat is slightly higher, perhaps due to more pronounced dilution (less C₃S initially implies a greater degree of reaction for the same amount of C₃S consumed). This behavior in the presence of a “non-preferred” filler is qualitatively similar to the heat response noted in presence of quartz fillers (FIG. 3). However, in addition to these interfacial effects, limestone fillers can contribute carbonate anions to the pore solution, which can subsequently be incorporated within the C—S—H gel. This kind of uptake likely occurs through ion-exchange reactions that release hydroxyl ions from the C—S—H to preserve charge neutrality. When carbonate incorporation is allowed by this kind of reaction (Eq. 19), the accelerating effect of the limestone is largely unchanged at the beginning because it still offers the same preferential nucleation sites, as shown in FIG. 10(b). However, as more C—S—H is formed through hydration, progressively more ion exchange can occur. OH⁻ ions released by the exchange reaction increase the driving force for C—S—H growth, by pH elevation, as compared with the driving force that evolves without CO₃²⁻ sorption. The result is an enhanced degree of reaction at later times. These conclusions can be further refined subject to more accurate experimental characterization of the carbonate uptake in the C—S—H. Nevertheless, the simulations do indicate that a chemical effect driven by CO₃²⁻ ion sorption, in addition to a preferential nucleation effect, is responsible for enhanced hydration in cements-containing limestone fillers. This ion sorption response is not reproduced in the nominally inert quartz systems due to the inability of the silicate species to induce ion-exchange reactions with the C—S—H.

Mechanistic Explanations of Accelerations in Cement Hydration Induced by Mineral Fillers:

The outcomes of this example provide new insights into the influence of mineral fillers on accelerating the rate of reactions in cementitious materials. Simulations performed using nucleation and growth models and stochastic reaction-transport models indicate that the acceleration is produced by a combination of factors: (1) the filler fineness, (2) interfacial properties, and (3) ion sorption/exchange effects. First, an increase in the filler fineness (solid surface area) accelerates hydration, but a proper balance ensures that aspects related to agglomeration, water trapping, and surface area saturation do not detrimentally influence the system response. The second factor in determining filler effects is the collection of the interfacial properties of the cement and the filler material, which can determine the extent and distribution of the nucleating hydration products. The energy barrier for heterogeneous nucleation on a surface is related to that for homogeneous nucleation of the same phase according to:

$$\begin{matrix} Δ {G}_{HET} = Δ {G}_{HOM} \cdot φ ( \text{?} ) \\ = ( \frac{16 π \text{?} {V}_{M}^{2}}{3 Δ {μ}^{2}} ) \cdot {[ \frac{( 2 + cos θ ) {( 1 - cos θ )}^{2}}{4} ]}^{\text{?}}\end{matrix} \\ cos θ = \frac{{γ}_{SL} - {γ}_{PS}}{{γ}_{PL}} \\ \text{?} \text{indicates text missing or illegible when filed} \qquad ( 22 )$$

where ΔG_{HET} is the energy that drives nucleation, applicable for the heterogeneous or homogenous case, Δμ=RT ln(1+S) describes the supersaturation level with respect to the precipitating phase, R is the ideal gas constant, S is the saturation index of the precipitate in solution described previously, V_{M} is the molar volume of the precipitate, γ_{SL} is the substrate liquid-specific interface energy (J/m²), γ_{PS} is the precipitate-substrate-specific interface energy (J/m²), γ_{PL} is the precipitate-liquid-specific interface energy (J/m²), θ is the thermodynamic contact angle, φ(θ) is an activity factor (indicative of wetting, adhesion, or surface affinity), which ranges between [0,1], n is a constant (n=0.33 for cap-shaped nuclei), and the subscripts P, L, and S indicate the precipitate (C—S—H), liquid, and solid substrate (limestone (l), quartz (q), or cement/C₃S (c)), respectively. Provided that ΔG_{HOM} for C—S—H precipitation remains fairly constant, Eq. (22) indicates that C—S—H nucleation on quartz particles would be opposed by a greater energy barrier than on limestone if the specific free energy of bonding with C—S—H, γ_{SL}γ_{PS}, is more positive for quartz than for limestone. This would be true if the bare limestone liquid interface has a greater average specific energy than quartz, or also if γ_{PS,1}<γ_{PS,q} (the C—S—H/limestone interface has a lower specific interface energy than the C—S—H/quartz interface). Datasets support the hypothesis that calcite (limestone) would provide a lower nucleation energy barrier for C—S—H nucleation than quartz.

The third factor that can influence reaction rates is the possible participation of dissolved species, liberated from the filler, in altering the course of hydration, either by precipitation of phases or by ion sorption reactions. Dissolved carbonate, in the presence of limestone, can impede the transformation of ettringite into monosulfate after gypsum is depleted because a carboaluminate phase is stabilized at the expense of monosulfoaluminate. But this is likely a small effect, due to lesser CO₃²⁻-Afm formation at early ages. The latter case (of ion exchange) is relevant, as charge compensation which follows sorption of CO₃²⁻ ions on the C—S—H is expected to lead to the release of OH⁻ species which elevates the pH and hence the driving force for continuing/onward hydrate growth. This point provides insights on compositional guidelines which may be used to infer the impact of fillers on hydration. Based on the above discussion, it is clarified that limestone is a superior acceleration agent than quartz, even at equal AM values (surface area), because both its favored interfacial properties and its ability to induce CO₃²⁻ sorption can enhance the rates of both nucleation and growth of the cementitious hydration products at early ages.

Conclusions:

This example describes the influence of mineral fillers on accelerating the rate of hydration reactions in cementitious materials. Simulation results are used to quantitatively interpret the role of dilution and the filler's characteristics on rates of reactions. Aspects of surface area, interfacial properties, and ion-exchange (sorption) reactions are distinguished and analyzed separately in terms of their influence on hydration rates. The results indicate that limestone is more effective than quartz (and certain other fillers) as an accelerant due to its interfacial properties and its ability to participate in ion-exchange reactions. Overall, the results shed new light on the filler effect and point the way to improved methods to better analyze, quantify, and screen minerals in terms of their ability to serve as cement replacement agents. Information of this nature is relevant in the context of enhancing prevailing cement replacement levels in concrete, the evaluation of new and superior fillers, and proportioning low-cement content concretes, such that mechanical property development and concrete durability could remain largely unaffected, in spite of reductions in the cement content.

### Example 2

### A Comparison of Intergrinding and Blending Limestone on Reaction and Strength Evolution in Cementitious Materials

The use of powdered limestone is a promising approach to reduce the clinker factor of Portland cements. Recent regulatory actions in the United States and Canada have allowed for Portland cements to contain up to 15% limestone (mass or weight basis). Cement replacement by limestone can be achieved by: (1) intergrinding cement clinker and limestone through the cement production process or (2) blending the cement and graded limestone powders through the concrete batching-mixing process.

While both methods of cement replacement (intergrinding or blending) appear similar, it is unclear if similar rates and extents of reaction and property development can be achieved by both methods, so long as the clinker composition and surface areas (fineness) of the solid phases are similar. This aspect is important to understand, if from an industrial concrete proportioning perspective, limestone blended formulations can be constituted to display performance similar to interground limestone cements. To understand these aspects of limestone replacement and binder fineness in more detail, this example evaluates interground and blended limestone binders to evaluate if there is a method to establish reaction and property (strength) similarity between these materials. Specifically, this example evaluates cement pastes containing interground and blended limestone in terms of their hydration and strength evolution behavior. Experiments and numerical simulations performed within a BNG model indicate that the reaction response of interground cements can be achieved or exceeded by blended systems, depending on the characteristics of the cement and the limestone used, such as Type I/II, Type III or blend of Type I/II and Type III. Thus, by adjusting the cement or limestone fineness, blended systems can be proportioned to display strengths which are superior to the interground case at early ages. However, by later ages binders show similar strengths. The results do suggest that for replacement levels up to 15% (weight basis), intergrinding or blending are both viable strategies to reduce the clinker factors of Portland cements, while maintaining early-age properties similar to pure cement formulations.

Materials, Mixing Procedures, and Methods:

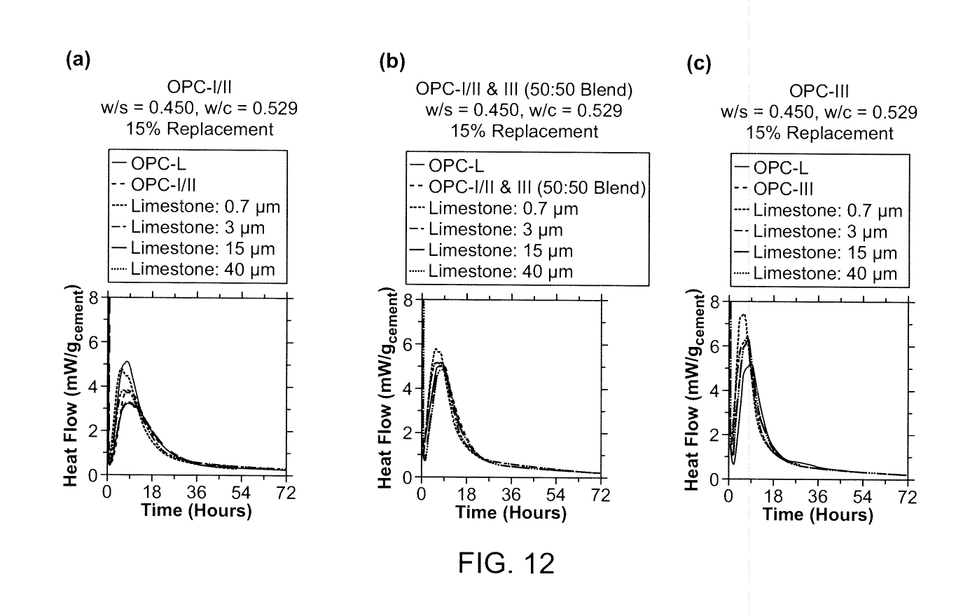

Three commercially available cements (designated ordinary Portland cement (OPC)) were used in this example. The phase composition of the cements is provided in Table II. The limestone powders used are (nominally pure) commercially available, particle size classified products produced by OMYA A.G. Particle size distributions (PSDs, FIG. 11) of the solids were measured using static light-scattering using isopropanol and sonication for dispersing the powders to primary particles. The uncertainty in the scattering measurements was determined to be about 6% based on measurements performed on six replicate powder samples, assuming the density of the cement and limestone to be 3150 kg/m³ and 2700 kg/m³ respectively.

TABLE II
The Estimated Phase Compositions of the Cements used in Example 2

| ID | Phase | Mass % | ID | Phase | Mass % | ID | Phase | Mass % |
|---|---|---|---|---|---|---|---|---|
| OPC I/II | C₃S | 59.1 | OPC-L | C₃S | 63.8 | OPC III | C₃S | 62.3 |
| | C₂S | 15.9 | | C₂S | 8.9 | | C₂S | 10.3 |
| | C₃A | 3.7 | | C₃A | 7.1 | | C₃A | 3.9 |
| | C₄AF | 10.8 | | C₄AF | 9.5 | | C₄AF | 14.2 |
| | Na₂O equivalent | 0.40 | | Na₂O equivalent | 0.70 | | Na₂O equivalent | 0.50 |
| | Limestone | ≈0-5%^{a} | | Limestone | ≈6-15%^{b} [30] | | Limestone | ≈0-5%^{a} |

Cementitious paste mixtures were prepared using de-ionized (DI) water at a fixed water-to-solids weight ratio (w/s=0.45) as described in ASTM C305. It should be noted that, in the case of blended mixtures, the dry cement and limestone powders were homogenized, prior to mixing in a planetary mixer. To understand the role of the limestone introduction mode (intergrinding or blending) and limestone fineness, for the Type I/II and Type III cements, the cement content was reduced by the maximum permissible limit: to permit 15% (weight) replacement of cement, by limestone powders of varying particle sizes while the interground cement (designated OPC-L) was evaluated as supplied. Further experiments were performed by composing blended binders where the OPC fraction (85%, by weight) was constituted as a 50:50 (weight) blend of Type I/II and Type III OPCs, to achieve a cement fineness midway between the individual OPC types, while the residual weight fraction contained limestone powders of varying fineness, added to achieve a 15% (by weight) cement replacement level. While the weight-based replacement of cement does alter the volumetric water-to-solid ratio (w/s_{V}) of mixtures containing limestone (due to density differences amongst the solid phases), the level of change is small, ranging from 1.42 for the plain cement paste, to 1.38 for the 15% limestone mixtures. This level of change would then correspond to a change in the weight-based water-to-cement ratio (w/c_{M}) for a plain cement paste from 0.45 to 0.44.

The influence of limestone additions (cement replacement) on the solid surface area of the system is described using an area multiplier (AM, unitless) as shown in the following equation:

$$AM = \frac{100 * \frac{( r \text{?} \text{?} ) \text{?} ( ( 100 - r ) \text{?} \text{?} )}{( ( 100 - r ) \text{?} \text{?} )}}{100} \\ \text{?} \text{indicates text missing or illegible when filed} \qquad ( 23 )$$

where r (weight %) is the percentage replacement of cement by limestone and SSA_{C} and SSA_{F} (m²/g) are the specific surface areas of the cement and limestone, respectively—calculated using the particle size distribution of the powder materials, while assuming spherical particles. It should be noted that surface areas calculated within this approximation can be underestimated by a factor of about 1.6-1.8, given the angular nature of the cement and limestone particles.

The influence of cement replacement on the rates of reactions was tracked using isothermal conduction calorimetry. A Tam Air isothermal calorimeter (TA Instruments, DE, USA) was used to determine the heat evolved during hydration at a constant temperature condition (25° C.). The thermal power and energy measured were then used to assess the influence of powder additions on the reaction kinetics and the cumulative heat release of the cement paste.

The compressive strength of cubic (50 mm×50 mm×50 mm) specimens cured at 25±1° C., in a sealed condition was measured as described in ASTM C109 at 1, 3, 7, and 28 days for all the mixtures with the exception of the Type III plain cement paste and the 15 μm, 15% limestone containing Type III (blended) paste for which datasets are available at 1, 3, and 7 days and 1 day respectively. Also, it should be noted that strength determinations were not carried out for the 50:50 OPC blend mentioned above, which was evaluated solely in terms of its reaction rate behavior. The compressive strength reported is typically the average of three specimens cast from the same mixing batch. The coefficient of variation (CoV) in the measured strength was determined to be about 10% for samples cast from the same batch.

### Experimental Results and Discussion

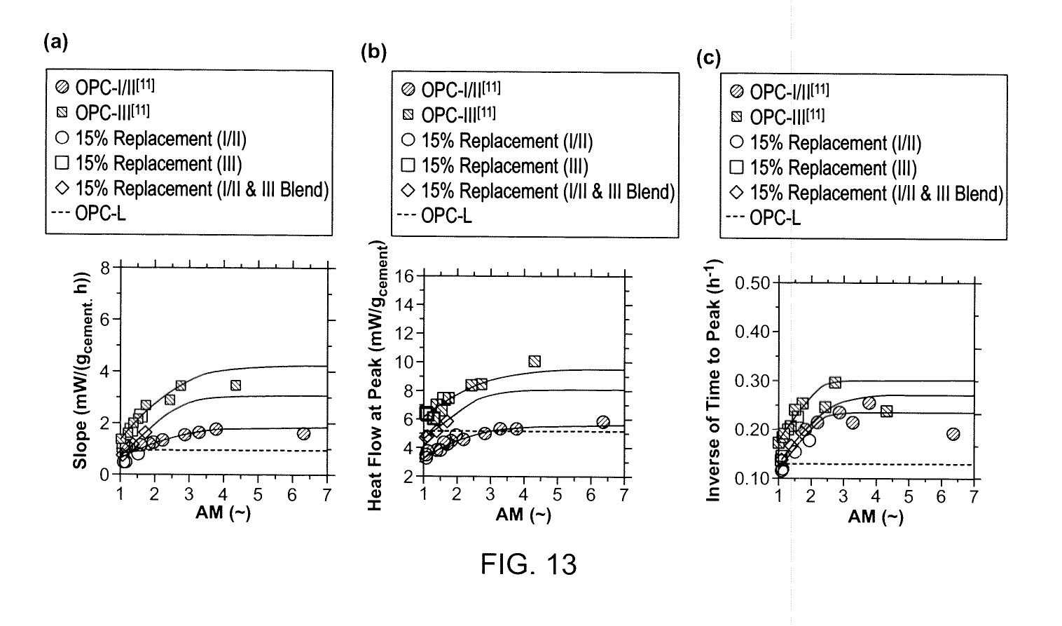

FIG. 12 shows the influence of cement type (fineness) and limestone particle size on the rate of hydration reactions. It is noted that, in general, an increase in the cement fineness, limestone fineness, or cement replacement level acts to increase the rate of chemical reactions. This increase (acceleration) manifests as a left-shift of the heat flow curve and elevation in the heat flow value at the main peak. While this effect is somewhat influenced by the chemistry of the mineral filler and its interfacial/compositional properties, this response can be understood as an increase in the fineness of either, or both, the cement and the limestone that leads to an increase in the surface area available for reactions, resulting in an acceleration in hydration.

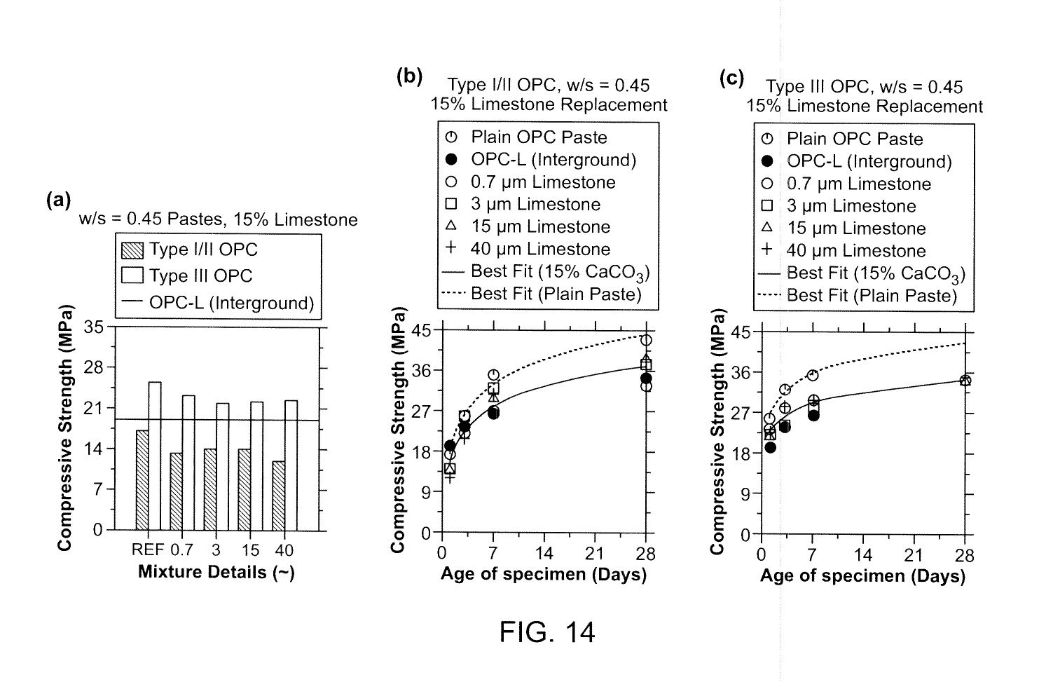

To better quantify the acceleration in binder hydration due to limestone additions, calorimetric parameters including: (a) the slope during the acceleration period, (b) the heat flow at the main peak and (c) the inverse of time to achieve the heat peak are plotted as a function of the AM (FIG. 13). The trends indicate that rates of reactions are enhanced in proportion with the AM of the system, namely as a function of the cement and limestone fineness. Thus, the reaction rates of the 50:50 OPC blends and the OPC-L mixtures lie intermediate between the reaction response of the Type I/II (lowest surface area) and Type III (highest surface area) OPCs, for mixtures prepared at the same or corresponding dilution (w/s). While these results do suggest that an ever progressive increase in the AM will amplify the reaction rate, this is an effect of diminishing returns. For example, as is clarified by FIG. 13, indeed increasing the AM does act to increase reaction rates, but this effect is applicable up to AM≦4 after which little or no further acceleration in reactions is noted. This diminishing role of the AM on the reaction rate (parameters) likely results from either or both: (1) the enhanced agglomeration of fine solid particles, which would act to reduce their exposed surface area and trap water in flocs inducing a less than expected acceleration in reactions and (2) a surface area saturation effect, where for AM≧4, the available surface area is more than that required for reaction of the available quantity of cement, resulting in a plateau in the measured reaction parameters.

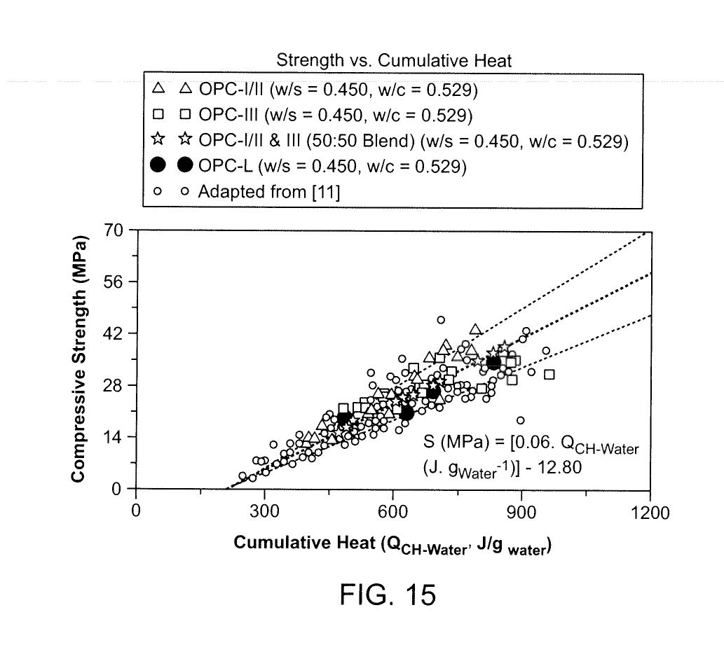

To examine the influence of the limestone addition mode (intergrinding or blending) on the mechanical properties, compressive strength determinations were carried out. It is noted that the plain cement pastes (w/c=0.45) for both the Type I/II and Type III OPCs show the best strength behavior, being around 10% stronger than the limestone-containing mixtures. The exception is at 1 day, when the OPC-L mixture develops a slightly higher strength than the Type I paste (FIG. 14a). For all the limestone mixtures, and in accordance with the reaction evolution trends, the compressive strength at 1 day was noted to scale as shown in FIG. 14a where: Type III>OPC-L>Type I/II, as linked to the finenesses of the cement and the limestone contained in each formulation. However, by later ages (e.g., 28 days), it is noted that all limestone-containing pastes show compressive strengths that are similar to each other (FIGS. 14b and c). It should be noted that this example does not consider differences in microstructural packing that may manifest, as the particle size of the limestone is changed. However and broadly, the trends indicate that early-age strength evolves in relation to the binder fineness, with the Type III mixtures showing higher early age strengths than similarly constituted Type I/II OPC systems, and the OPC-L system showing intermediate strengths—an areal function of overgrinding of the cement clinker and limestone through production. These results indicate that, in blending operations, the partial or complete use of a fine cement would be a viable method to achieve elevated early strengths.

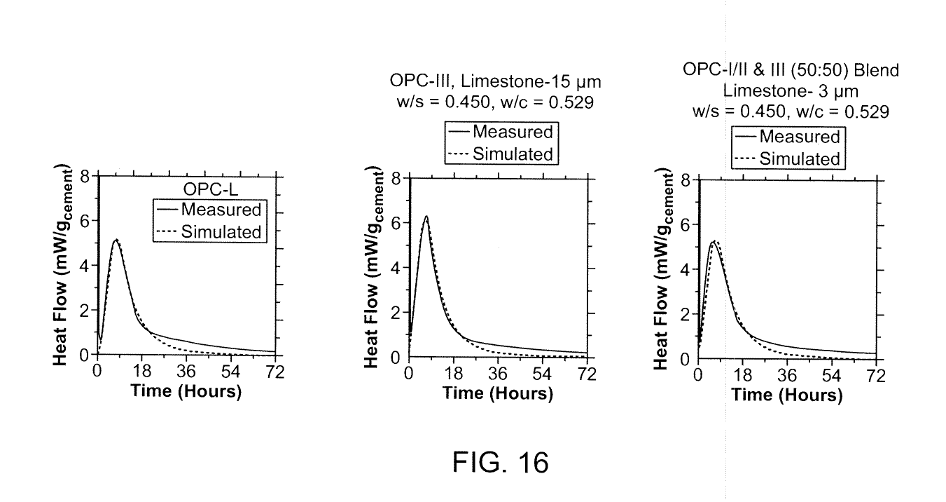

The evolution of the compressive strength has been shown to be linearly related to the release of heat through hydration. It should be noted that the measured heat is normalized by a mixture's initial water content (volume basis, assuming the density of water, ρ_{W}=1 g/cm³), as the initial volumetric water content is a measure of the initial porosity of the system—space that can be progressively filled by the hydration products towards achieving better properties. To better quantify this relationship for both blended and interground systems, heat-strength datasets for the current mixtures were plotted alongside a larger dataset previously developed for blended, OPC-limestone mixtures made using limestone powders having varying median particle sizes, where the OPC content was reduced in 10% increments, from 0%-to-50% (by weight) as shown in FIG. 15.

As noted in FIG. 15, the heat-strength relationship of the current set of mixtures is in good agreement with previously developed datasets. A linear correlation of this nature is of note in that it indicates that, irrespective of limestone addition mode (intergrinding or blending), measures of heat release through isothermal calorimetry can be used to infer the evolution of mechanical properties in these binders. It is also noted that the estimated strength of the 50:50 OPC blends lies intermediate to the Type I/II and Type III OPC mixtures, and is essentially similar or slightly superior to the OPC-L mixtures—an expected outcome based on the intermediate AM (Table III) and level of reaction evolution noted in the 50:50 OPC blended systems as shown in FIGS. 12 and 13. This relationship then clarifies that, for any given mixture, correspondence or similarity in terms of cumulative heat release through hydration, when normalized by the water content, indicates mechanical property correspondence or similarity between the mixtures.

TABLE III
Measured d₅₀ and Calculated Specific Surface Area
(SSA) Values for the Cement and Limestone Powders,
as Determined using Static Light-Scattering.

| | ASTM C150 OPC | | Size classified limestone | | | |
|---|---|---|---|---|---|---|
| | d₅₀ | SSA_{C} | | | d₅₀ | SSAᵣ |
| Cement ID | (μm) | (m³/kg) | limestone ID | | (μm) | (m²/kg) |
| Type I/II | 9.83 | 486.00 | L-0.7 | μm | 1.40 | 2592.10 |
| OPC-L | 6.76 | 601.50 | L-3 | μm | 2.98 | 1363.20 |
| Type III | 5.61 | 780.27 | L-15 | μm | 14.87 | 399.20 |
| Type I/II and III | — | 633.14 | L-40 | μm | 40.10 | 228.60 |
| 50:50 blend | | | | | | |

Boundary Nucleation and Growth Model for Cement Hydration:

Classical and modified forms of boundary nucleation and growth (BNG) models can be applied to describe the hydration of cement systems. These models simulate reactions as a nucleation and growth process that starts at the solid phase boundaries. In these models, a single product of a constant density is assumed to nucleate and its growth is treated as the rate-controlling mechanism that determines the kinetics of the reaction. BNG models can be formulated with a variety of assumptions for the reaction rate-controlling mechanisms, including nucleation site saturation, product growth control, and continued nucleation of products. This example applies a modified form of a BNG formulation to better interpret the influence of the limestone addition mode (blending or intergrinding) on the kinetics of reactions using Eqs. (24), (25), (26a), (26b), (27), and (28) as set forth below:

$$X = 1 - exp [ - 2 {a}_{BV} \text{?} ( 1 - exp ( - \text{?} ) ) d y ] \\ \text{?} \text{indicates text missing or illegible when filed} \qquad ( 24 )$$

where X is the volume fraction of the reactant transformed to product, Gₒᵤₜ is the isotropic outward growth rate of the product phase, y is a variable of integration, a_{BV} is the boundary area per unit volume, t is the simulation time (h), and A_{f} is the extended area of the transformed product phase described in the following equations:

$$\begin{matrix}\text{?} = π [ {I}_{density} \cdot {G}_{par}^{2} \cdot ( \text{?} - \frac{{y}^{2}}{\text{?}} ) + {I}_{rate} \cdot {G}_{par}^{2} \cdot ( \frac{\text{?}}{3} - \frac{\text{?}}{\text{?}} + \frac{\text{?}}{\text{?}} ) ] & ( 25 ) \\ \text{?} \text{?} = 0 if ( y < \text{?} ) & ( 26 a ) \\ Here , ( \text{?} = ( t - {t}_{0} ) ) \\ \text{?} \text{indicates text missing or illegible when filed} & ( 26 b )\end{matrix}$$

where I_{density} (μm²) is the nucleation density of the product, that is, the starting number of supercritical nuclei per unit surface area, Iᵣₐₜₑ (μm⁻²/h) is the nucleation rate, Gₚₐᵣ (μm/h) is the growth rate parallel to the particle surface, and Gₒᵤₜ (μm/h) is the outward growth rate, perpendicular to the particle surface. The cumulative heat evolved by reaction of the cement is computed using a scaling parameter, A (kJ/mol), as shown in the following equation:

$$Rate of heat evolution ( \frac{d H}{d t} ) = A \text{?} ( \frac{100}{100 - r} ) \frac{d X}{d t} \\ \text{?} \text{indicates text missing or illegible when filed} \qquad ( 27 )$$

where r (%) is the (weight) percentage replacement level of filler which accounts for the effects of dilution (a reduction in reactive cement content). In addition, since the simulations begin at the end of the induction period which varies slightly from one mixture to another, the simulation time is mapped to real time by using a parameter t₀ to designate the time at which the induction period ends as shown in Eq. (26b). As such, the free variable t₀ is assigned an increasingly positive value when the simulated curve is to be left-shifted, and an increasingly negative value when the induction period is lengthened, and the simulation curve is right shifted along the temporal (time, x) axis. The boundary area per unit volume, a_{BV} (μm⁻¹), is calculated by adding the surface areas of both the cement and limestone filler and dividing by the volume of the overall system (total solid content plus water):

$$\text{?} = \frac{{SSA}_{cement} \text{?} \text{?} \text{?} {ρ}_{cement} \cdot ( 100 \cdot {f}_{cement} )}{\text{?}} \\ \text{?} \text{indicates text missing or illegible when filed} \qquad ( 28 )$$

where f_{cement} (unitless) is the initial volume fraction of cement, ρ_{cement} is the density of the cement (3.15 g/cm3), V_{free} (μm³) is the initial volume of water present in the system, and SSA_{cement} is the specific surface area of the cement. The parameter a_{factor} (unitless) acts as a free variable representing a “virtual AM” used in the simulations. For all simulations, the values of Iᵣₐₜₑ, Gₒᵤₜ, and Gₚₐᵣ are drawn from prior simulations of OPC/limestone blends and are thus noted as 0.00 μm⁻²/h, 0.03 μm/h, and 4.00 μm/h, respectively. It should be noted that the assignment of Iᵣₐₜₑ=0.00 μm⁻²/h would correspond to the case of nucleation site-saturation, implying that growth of the product phase begins from nuclei that are initially present, or form at very early ages such that little or no further nuclei are permitted to form after the initial nucleation burst. While site saturation was the assumption considered in this example, impositions of a constant nucleation rate (Iᵣₐₜₑ>0) and product growth rate control also can be used to simulate cement hydration. In all the equations above, f_{cement} (unitless) and a_{BV} (μm⁻¹) serve as input variables while A (kJ/mol), I_{density} (μm⁻²), a_{factor} (unitless), and t₀ (h) remain free (fitting) variables. To fit the response of the limestone containing pastes, a simplex method was applied as follows: (1) I_{density} and a_{factor} are varied within defined constraints to match the upslope and the time of peak through the acceleration regime, (2) the parameter A is scaled to match the amplitude corresponding to the heat flow rate at the time of the main peak, and (3) t₀ is adjusted to shift the simulated heat flow response to the right or to the left to temporally match the measured heat response.

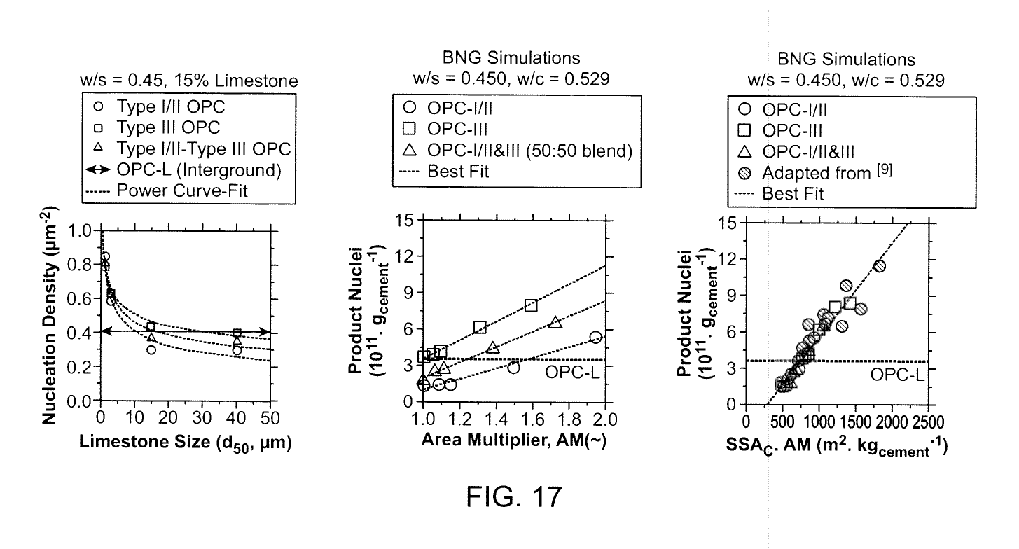

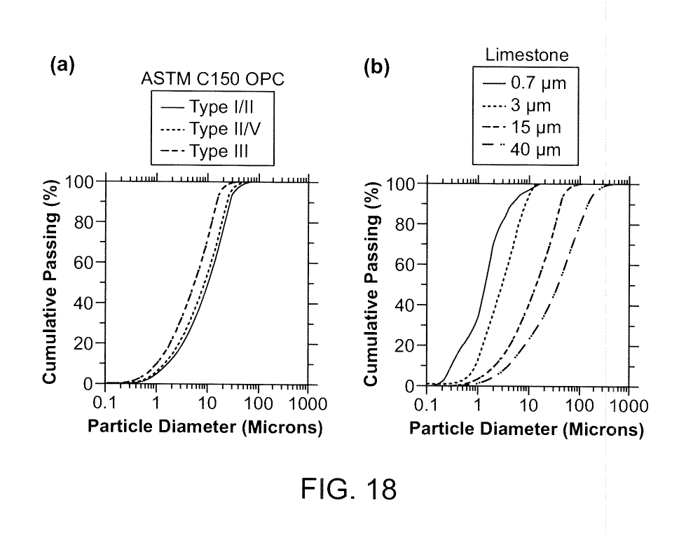

FIG. 16 and Table IV show representative best-fit results and parameters used in simulations for the interground and limestone blended systems. The parameter optimizations indicate that A and t₀ are varied within a small range between (57-68 kJ/mol) and (0-1.13 h) respectively, but no systematic trend was found in their variation. The values of a_{factor} (virtual AM) and I_{density} (nucleation density) both increase with decreasing limestone size (and surface area), indicating that fine limestone is a better acceleration agent than coarse limestone (FIG. 17c). Information of particle size dependence can be correlated with the calculated nucleation density as shown in FIG. 17a, to determine how reaction evolution in blended Type I/II and Type III OPCs can be equated to any interground systems. For example, FIG. 17 indicates that reaction correspondence or similarity to the interground system can be achieved by blending (by weight), 15% limestone of progressively increasing fineness, as the OPC fineness decreases (a finer limestone for a Type I/II OPC and a coarser limestone for a Type III OPC respectively). This result is intuitively reasonable, as actions of this nature would act to boost the solid surface area of the Type I/II mixtures, and depress the surface area of the Type III mixtures as a method to equate reaction rates. As can be appreciated, the blended OPC (50:50, Type I/II and Type III) mixtures lie between the two extreme cases a function of their intermediate solid surface area.

TABLE IV
Parameters used to Simulate the Hydration Response of Interground and Blended
Paste Systems using a Modified BNG formulation.

| | | | | | | | |
|---|---|---|---|---|---|---|---|
| Batch # | A (kJ mol⁻²) | Iᵣₐₜₑ (μm⁻²h⁻¹) | I [symbol] (μm⁻²) | G [symbol] (μm h⁻¹) | G [symbol] (μm h⁻¹) | SSA_{cement} · a_{factor} (m² kg_{cement} ⁻¹) | tₒ ( [symbol] ) |

OPC-I/II

| | | | | | | | |
|---|---|---|---|---|---|---|---|
| L-0.7 μm | 59.11 | 0.00 | 0.840 | 0.03 | 4.0 | 6481.00 | −0.21 |
| L-3 μm | 60.71 | 0.00 | 0.585 | 0.03 | 4.00 | 4955.40 | −0.69 |
| L-15 μm | 56.74 | 0.00 | 0.300 | 0.03 | 4.00 | 4862.00 | −1.13 |
| L-40 μm | 57.39 | 0.00 | 0.298 | 0.03 | 4.00 | 4861.21 | −1.00 |

OPC-III

| | | | | | | | |
|---|---|---|---|---|---|---|---|
| L-0.7 μm | 68.24 | 0.00 | 0.787 | 0.03 | 4.00 | 10239.04 | −0.65 |
| L-3 μm | 60.32 | 0.00 | 0.625 | 0.03 | 4.00 | 9944.69 | −0.56 |
| L-15 μm | 63.03 | 0.00 | 0.435 | 0.03 | 4.00 | 9697.56 | −0.72 |
| L-40 μm | 64.47 | 0.00 | 0.404 | 0.03 | 4.00 | 9691.22 | −0.52 |

50-50 Blend of OPC-I/II and OPC-III

| | | | | | | | |
|---|---|---|---|---|---|---|---|
| L-0.7 μm | 61.56 | 0.00 | 0.812 | 0.03 | 4.00 | 8176.85 | −0.48 |
| L-3 μm | 61.04 | 0.00 | 0.604 | 0.03 | 4.00 | 7623.01 | 0.00 |
| L-15 μm | 60.37 | 0.00 | 0.367 | 0.03 | 4.00 | 7899.08 | −0.87 |
| L-40 μm | 59.55 | 0.00 | 0.351 | 0.03 | 4.00 | 7763.99 | −0.68 |

OPC-

[symbol]

| | | | | | | | |
|---|---|---|---|---|---|---|---|
| OPC-L | 60.18 | 0.00 | 0.406 | 0.03 | 4.00 | 8771.53 | −0.06 |

[symbol]

indicates data missing or illegible when filed

The fitting parameters a_{factor} and I_{density} were combined to calculate the number of supercritical product nuclei produced per gram of reactant as shown in Eq. (29). Here, product nuclei (g⁻¹_{cement}) denotes the number of supercritical nuclei produced per gram of cement reacted, and SSA_{Effective,Simulations} (m²/kg_{cement}) and SSA_{Effective,Measured} (m²/kg_{cement}) represent the simulated and measured values of surface area per unit mass of cement that is effectively available for the nucleation (and the onward growth) of the hydration products.

Product nuceli (#/g_{cement})=(SSA_{cement}·a_{factor})·I_{density}

where SSA_{Effective,Simulations}=SSAcement·a_{factor}

and, SSA_{Effectiv,Measured}=SSA_{cement}·AM (29)

The number of supercritical product nuclei produced per gram of cement reacted is plotted against the AM (FIG. 17b). For each cement type, increasing the available solid surface area, by the incorporation of size classified limestone (or reducing the OPC fineness), results in a linear increase in the number of nuclei that participate in reactions. This trend indicates that, all other parameters remaining equal, fine limestone by provisioning a higher solid surface area for a given mass is able to induce the formation of a larger number of supercritical hydration product nuclei which participate in reactions (higher I_{density} values, while Iᵣₐₜₑ remains fixed), an action which would enhance the formation of the reaction products, and thus accelerate early age reactions.

In addition to the nucleation density, the number of (supercritical) product nuclei estimated by the simulations can also be plotted as a function of the AM, and the SSA_{Effective,Measured} (Eq. (29)) as shown in FIGS. 17b and c. It is noted that the discrete trend-lines noted in FIG. 17b collapse onto a single master curve in the latter case. This result indicates a linear dependence between the number of product nuclei produced through hydration (7.70×10¹⁴ nuclei per unit quantity of cement) and the surface area of the system (largely independent of the limestone addition mode), which is a function of the specific surface area of the constituent phases (OPC and limestone). This result indicates that blended systems can be designed to have corresponding or similar reaction kinetics (and thus strength evolution behavior) as interground systems, and vice versa, by selecting their effective surface areas to be similar or identical, by tailoring one or more of: (a) OPC fineness, (b) limestone fineness and (c) the extent of OPC replaced by limestone filler.

Conclusions:

This example has compared and contrasted the evolution of hydration and strength in interground and blended limestone systems. By the careful integration of experiments and simulations, it is demonstrated that reaction and by extension strength evolution in these systems are, for similar OPC chemistries, broadly a function of the OPC and limestone fineness, and the extent (weight fraction) of OPC replacement by limestone. This result indicates that similarly performing systems can be proportioned by either blending or intergrinding OPC and limestone so long as the: (1) level of OPC replacement is similar and (2) either, or both, OPC and the limestone fineness can be tailored to achieve similar solid surface areas in the system. Applicability of this approach can be bounded by: (1) dispersion and agglomeration when the limestone (or OPC) particle size is sufficiently small, and the level of cement replacement large and (2) gel-space ratio (quantity of hydration product (C—S—H) formed from the hydration reactions, as beyond a certain point, if insufficient hydration product formation occurs, strength development can be suppressed. It should be noted that a single strength-heat master curve (SHMC) capable of describing strength evolution in both interground and blended binder systems allows estimations of properties in mixtures constituted by either method. It also should be noted that the relationship shown in FIG. 15 applies to plain and binary mixtures constituted using broadly inert fillers. Depending on the reactivity of a filler, the slope of the best-fit line relevant to the SHMC may be altered, thus altering the mathematical form of the relationship sketched in FIG. 15.

### Example 3

### Methods to Estimate the Influence of Limestone Fillers on Reaction and Property Evolution in Cementitious Materials

Commercial interest in sustainable cementing materials is driving efforts to reduce the use of cement in concrete. Limestone fillers are a promising direction towards achieving such cement use reductions. In spite of increasing filler use, little information is available to rapidly estimate the influences of limestone fillers, and more importantly filler fineness on reaction and property development. This example develops a model to predict the effect of particle size classified limestone on hydration reactions and compressive strength development. The model builds on a relativistic basis, such that enhancements and alterations in reactions and properties are described in relation to a given control (pure cement) mixture. The prediction model considers aspects such as: (1) accelerations in reactions, (2) changes in inter-particle spacing as linked to the limestone filler's fineness and (3) a porosity increase with increasing cement replacement. The predictive power of the approach is demonstrated for a variety of mixtures composed using three ASTM C150 compliant cements and forwards a basis for developing mixture proportioning strategies, such that apriori estimations of the mixture response (reaction rate and mechanical properties) can be used to optimize binder proportioning and thus strategize new methods to limit cement use in concrete construction applications.

Specifically, this example sets forth relationships based on chemical and physical indicators which can be used to predict the influence of size classified limestone additions on hydration and strength development in these materials. Based on a large experimental dataset, the approach is developed and applied for three ASTM C150 compliant cements, for cement replacement levels ranging between 0-50% (by weight) by limestone filler. Special attention is paid to limestone as its ability to serve as a “mineral acceleration agent” advances opportunities to reduce the cement content in a binder, by accelerating hydration product formation at early ages. Thus the example advances: (1) strategies for concrete technologists to virtually estimate the influence of the cement replacement level and limestone fineness on reactions and property development and (2) provides a method to avoid time consuming, empirical mixture evaluations. The results have broad implications on refining mixture proportioning strategies, and introduce new approaches which can be used to proportion the next generation of binders with a reduced cement content.

Materials, Mixing Procedures, and Methods:

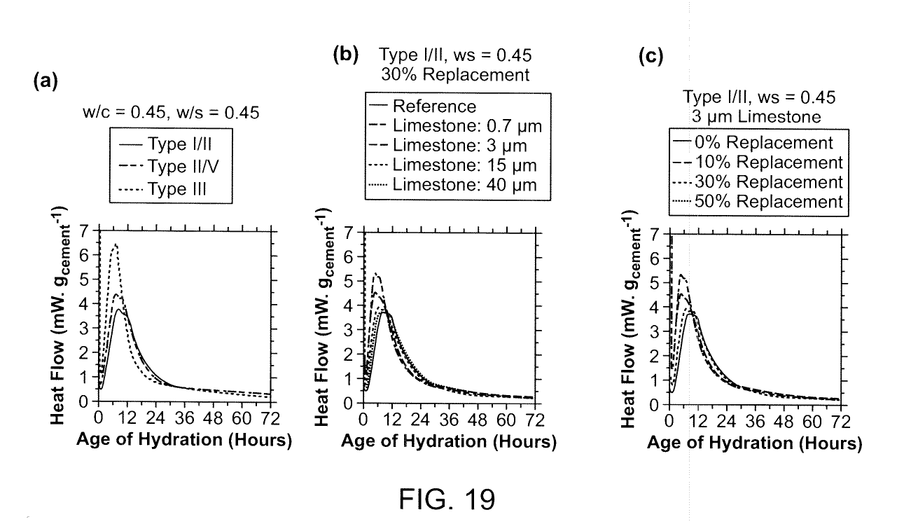

Three ASTM C150 compliant cements were used in this example. The phase compositions of the cements used are provided in Table V. The limestone powders used are nominally pure, commercially available, particle size classified products produced by OMYA A.G. The particle size distributions (PSD, FIG. 18) of all the solids were measured using light-scattering using isopropanol and sonication for dispersing the powders to primary particles. The uncertainty in the scattering measurements was determined to be about 6% based on measurements performed on six replicates assuming the density of cement and limestone to be 3150 kg/m³ and 2700 kg/m³ respectively. Cementitious paste mixtures were prepared using de-ionized (DI) water at a fixed water-to-solids weight ratio (w/s=0.45) as described in ASTM C305. To better understand the role of the limestone filler, the cement content was progressively reduced, by replacement in 10% increments, from 0-50% (weight basis) by limestone powders of varying median particle (d₅₀) sizes. Other mixtures were prepared with w/c corresponding to those obtained for the cement replaced systems, ranging from w/c=0.45-to-0.643.

TABLE V
Phase Compositions of the Ordinary Portland Cements used in Example 3

| ID | Phase | Mass % | ID | Phase | Mass % | ID | Phase | Mass % |
|---|---|---|---|---|---|---|---|---|
| OPC I/II | C₃S | 63.10 | OPC II/V | C₃S | 62.60 | OPC III | C₃S | 63.30 |
| | C₂S | 12.89 | | C₂S | 11.76 | | C₂S | 10.31 |
| | C₃A | 3.67 | | C₃A | 4.58 | | C₃A | 3.93 |
| | C₄AF | 10.83 | | C₄AF | 13.92 | | C₄AF | 14.22 |
| | Na₂O | 0.38 | | Na₂O | 0.55 | | Na₂O | 0.47 |
| | Equivalent | | | Equivalent | | | Equivalent | |

The influence of powder additions (cement replacement) on the solid surface area of the system is described using an area multiplier (AM, unitless) as shown in Eq. (30):

$$AM = \frac{100 + \frac{( r \cdot \text{?} ) + ( ( 100 - r ) \cdot {SSA}_{C} )}{( ( 100 - r ) \cdot \text{?} )}}{100} \\ \text{?} \text{indicates text missing or illegible when filed} \qquad ( 30 )$$

where r (weight %) is the percentage replacement of cement by limestone filler, and SSA_{c} and SSA_{F} (m²/g) are the specific surface areas of the cement and limestone respectively—calculated using the particle size distribution of the powder materials, while assuming spherical particles.

The influence of cement replacement on the rate of reactions was tracked using isothermal conduction calorimetry. A Tam Air isothermal calorimeter (TA Instruments, DE, USA) was used to determine the heat evolved during hydration at a constant temperature condition (25° C.). The thermal power and energy were used to assess the influence of powder additions on reaction kinetics and the cumulative heat release of the cementitious mixtures.

The compressive strength of cubic (50 mm×50 mm×50 mm) specimens cured at 25±0.2° C., in a sealed condition was measured as described in ASTM C109 at 1, 3, 7, and 28 days. The compressive strength value reported is typically the average of three specimens. The coefficient of variation (CoV) in the measured compressive strength was determined to be about 10% for samples cast from the same mixing batch.

TABLE VI
Nominal d₅₀ and Specific Surface Area (SSA) Values
for the Cement and Limestone Powders, Calculated using
their Measured Particle Size Distributions.

| | ASTM C150 OPC | | Size Classified Limestone | | | |
|---|---|---|---|---|---|---|
| | d₅₀ | SSA | | | d₅₀ | SSA |
| Powder ID | (μm) | (m²/kg) | Powder ID | | (μm) | (m²/kg) |
| Type I/II | 9.83 | 486.00 | 0.7 | μm | 1.40 | 2592.10 |
| Type II/V | 8.94 | 538.02 | 3 | μm | 2.98 | 1353.20 |
| Type III | 5.61 | 780.27 | 15 | μm | 14.87 | 399.20 |
| | | | 40 | μm | 40.10 | 228.60 |

The time of initial and final set of the paste mixtures was determined as described in ASTM C191 at 25±3° C. In the ASTM C191 standard, the single laboratory precisions are listed as 12 minutes (0.2 hours) and 20 minutes (0.33 hours) for the time of initial/final set respectively.

### Experimental Results and Discussion

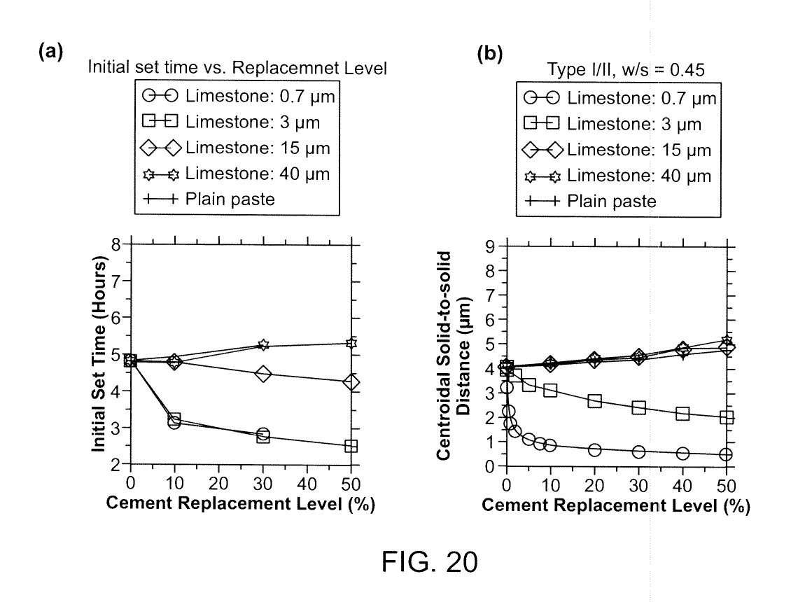

FIG. 19 shows the influence of: (a) cement type, (b) limestone particle size (fineness) and (c) the cement replacement level on the rate of hydration reactions. It is noted that, in general, an increase in the cement fineness, filler fineness, or filler content acts to increase the rate of chemical reactions. This increase (acceleration) manifests as a left-shift of the rate curve and elevation in the heat flow at the main peak. While this effect is somewhat influenced by the chemistry of the system and the nature of the filler agent, this response can be understood as an increase in the fineness of the cement or the limestone which increases the surface area available for reactions, resulting in an acceleration.

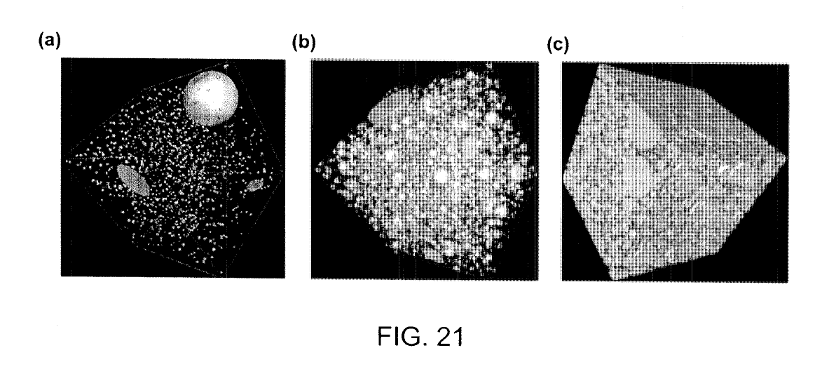

FIG. 20(a) shows the influence of the cement replacement level and limestone particle size on the time of initial set of paste mixtures. Fine limestone additions are an efficient way for decreasing the time of initial set of cementitious mixtures. This reduction of initial set time is a function of two effects including: (1) an acceleration in hydration reactions induced by limestone additions (as shown in FIG. 19) and (2) reductions in interparticle spacing produced (at a constant liquid content) by the addition of limestone, so long as the limestone is finer than the cement that it replaces.

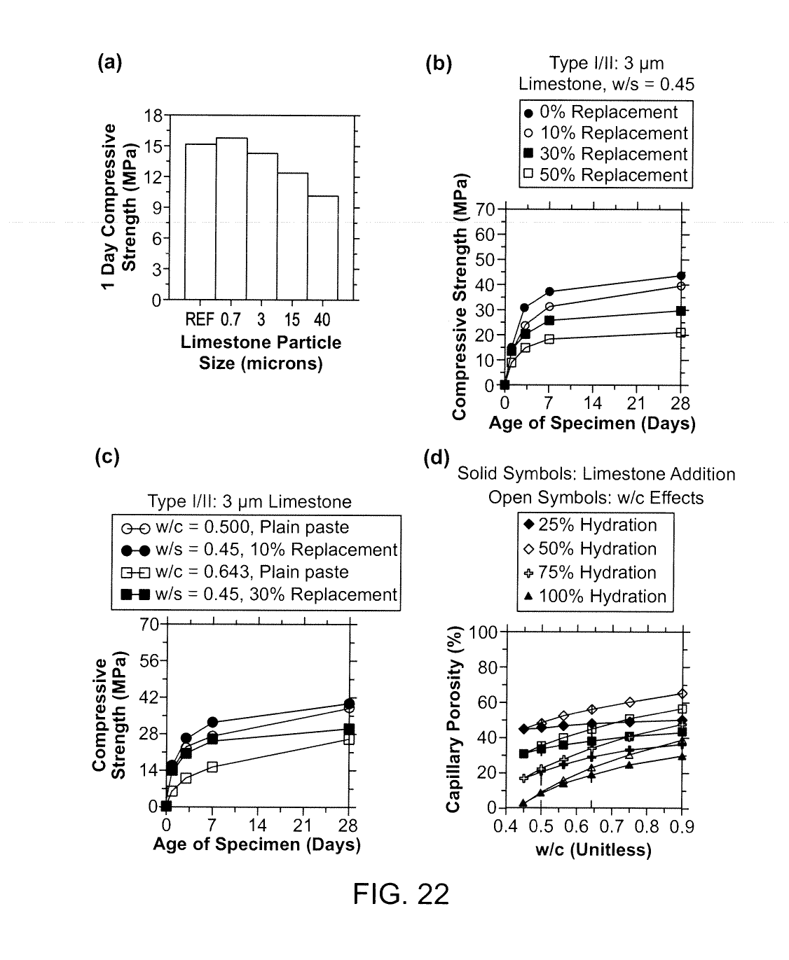

To study the particle spacing effect in detail, a microstructural stochastic packing method with periodic boundary conditions was implemented. This method, which uses the measured particle size distribution and volumetric fractions of materials as inputs (cement, limestone, and water), packs spherical particles in a 3D-REV (representative element volume) of 500×500×500 μm³. Microstructural generation and packing is permitted such that the minimum centroidal distance (C_{D}, μm, for size distributed particles) between two proximal particles is greater than the sum of their radii (C_{D}>r₁+r₂). The packing method packs the REV while iteratively analyzing and placing particles at random locations within the microstructure in relevance to two packing criteria: (1) the size (largest to smallest), and number of particles (information which is determined by the particle size distribution), within the constraint that particles do not contact and (2) the input volume fractions of the phases are satisfied, as described by the w/s of a given mixture (see FIG. 21). Once the sought packing is achieved, the mean solid-to-solid centroidal distance in the REV is calculated as follows: (a) 100 particles are randomly selected in the microstructure, (b) for each particle pᵢ, the solid-to-solid centroidal distance is computed with respect to all neighboring particles located within a distance of 5 μm away from the surface of pᵢ to identify its closest neighboring particles and (c) the mean solid-to-solid centroidal distance is calculated by averaging the centroidal distances calculated for all 100 particles. It should be noted that the selection of 100 random particles was made, as beyond this point the calculated centroidal distances between particles were noted to change very slightly, even if the number of analyzed solid particles was increased substantially. The mean solid-to-solid centroidal distance calculated as a function of the cement replacement level is shown in FIG. 20(b).

An examination of FIGS. 20 and 21 provides qualitative insights into the influence of mineral filler fineness and the cement replacement level on the trends observed in the time of initial set. Initial set is chosen as a time of relevance as this is an interval at which the solids are expected to be bridged (percolated) in 3D from contacts resulting from cement hydration—a point to be differentiated from surface to surface contacts between particles. It is noted that fine fillers (with a high specific surface area) decrease the time to achieve initial set at a given cement replacement level. This trend is observed to systematically invert as either the filler size or the water content (w/s) of the mixture is increased. For example, a paste with w/c=0.643 and a paste with w/s=0.45 (w/c=0.643), with 30% limestone replacement by 40 μm limestone both show a similar time of initial set (see FIG. 20a). This result indicates that the time of initial set is strongly correlated with the initial dispersion of the solid particles. Since fine fillers reduce (and coarse fillers increase) the interparticle spacing (see FIG. 20b; a consequence of better packing), this observation indicates that a decrease in the inter-particle spacing increases the propensity for 3D solid-percolation, by reducing the time/extent of hydration to achieve set an effect which is magnified by the mineral acceleration induced by limestone.

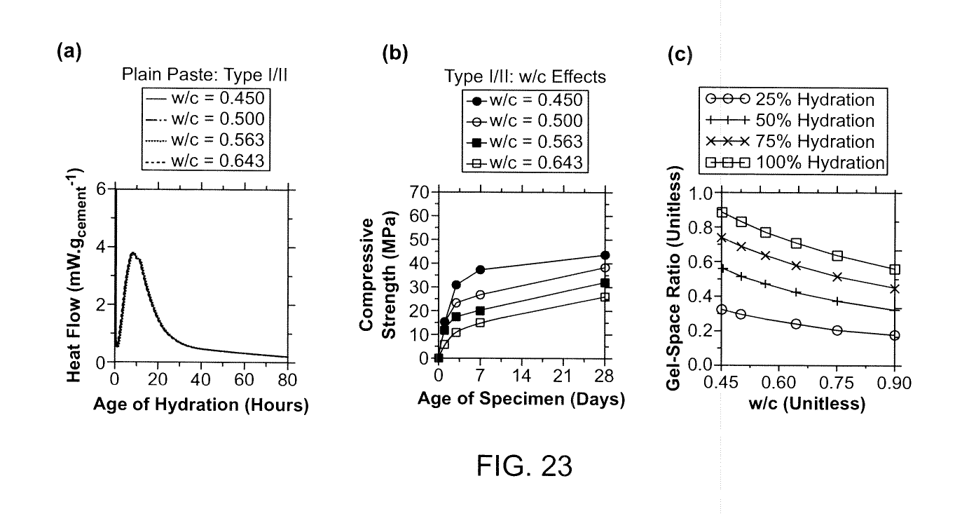

FIG. 22 shows the evolution of compressive strength for mixture parameters including: (a) the limestone particle size, (b) the cement replacement level and (c and d) the effects of w/c, for mixtures with and without cement replacement by limestone. From FIG. 22(a), it is noted that, at low replacement levels, the early age (1 day) strength is a function of the limestone particle size, with the highest strength (though slightly so) being produced by the 0.7 μm limestone filler, at 10% cement replacement. This observation can be understood as the acceleratory and packing effects of limestone, which improve with decreasing particle size and result in such a trend. This effect diminishes with increasing particle size, with the measured compressive strength decreasing accordingly. From FIG. 22(b), it is noted that the strength decreases with increasing cement replacement. This effect starts to attain increasing relevance, broadly, for cement replacement levels in excess of 10% (weight basis). Finally, FIG. 22(c) shows the influence of w/c correspondence for mixtures with and without cement replacement. Here, it is noted that mixtures which contain limestone show higher strengths up to 7 days, but the strengths measured at 28 days are more similar to the corresponding plain paste systems.

The w/c-strength response noted in FIG. 22(c) can be explained by considering the evolution of the capillary porosity in these mixtures which is shown in FIG. 22(d). As such, it can be rationalized that the higher strength of the limestone-containing pastes is an outcome of their lower capillary porosity, as caused by a higher solid loading. FIG. 22(d) also explains why mixtures composed at similar or identical w/c, both with and without limestone, show increasingly similar strengths with increasing age and thus increasing hydration. With increasing hydration, for the w/c evaluated, the ever diminishing difference in the capillary porosity (FIG. 22d) ensures that materials, with and without limestone, composed at similar w/c will, within certain bounds, exhibit similar strengths. This observation (similar strength after 28 days) may also be partially ascribable in that limestone is a much softer inclusion than either the unhydrated clinker phases, or the hydration products formed, and hence may be a weaker-link in the system at later ages.

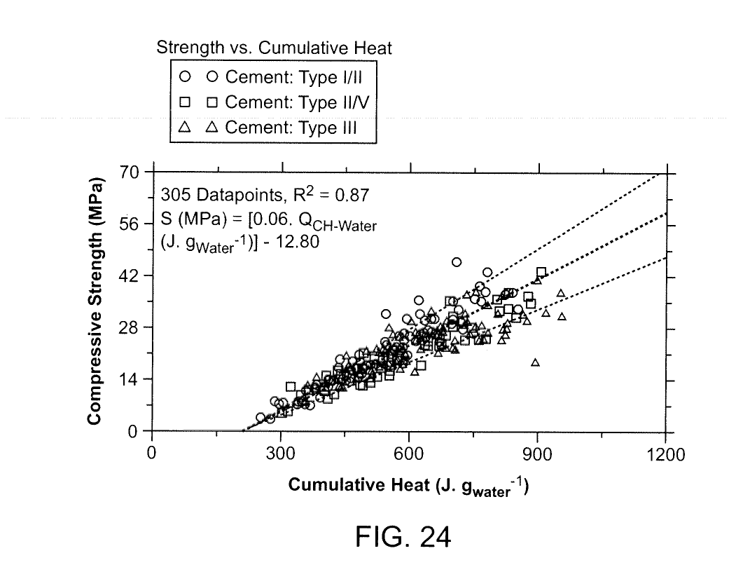

To describe the effects of a reduction in the cement content on reactions, a set of plain cement pastes were prepared with w/c ratios corresponding to the actual cement content in the systems with partial filler replacement levels ranging from 0-to-30% (weight basis, see FIG. 23a). It is noted that the calorimetry curves overlap, indicating similar reaction kinetics (rate/extent), suggesting that these systems hydrate similarly over the course of the experiment (over the first seven days). Results of this nature are applicable for pastes which hydrate (within certain bounds) in a water-sufficient system. In spite of correspondence in hydration, it is noted that the compressive strength of pastes decreases with an increase in their w/c (FIG. 23b). This result is in accordance with trends in the calculated gel-space ratio, where the gel-space ratio and the strength increase with decreasing w/c.

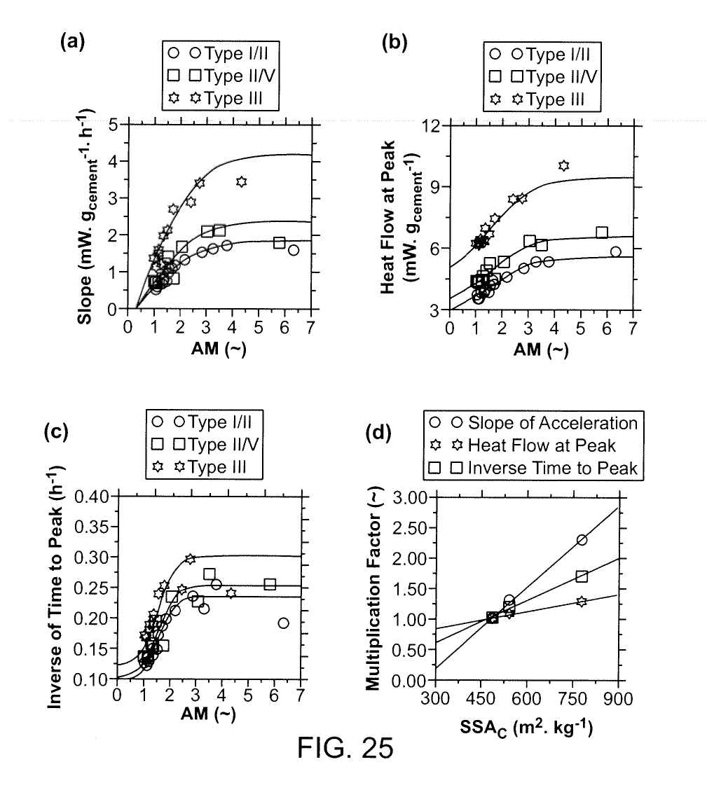

Heat evolution through hydration can be used as a measure of mechanical property (compressive strength) development in cementitious materials. To establish such correlations between the extent of hydration and the evolution of properties, the compressive strength values for all paste systems were cast as a function of the cumulative heat released normalized by the water content of the mixture at an age of 1, 3, 7, and 28 days as shown in FIG. 24. Here, it should be noted that the measured heat is normalized by a mixture's initial water content (weight or volume basis, assuming the density of water, ρ_{W}=1 g/cm³), as the initial water content is a measure of the initial porosity of the system (space that can be progressively filled by the reaction products to achieve better properties). As seen in FIG. 24, the heat-strength data-cloud is strongly correlated with a majority of data points lying within a ±20% bound of the linear best fit line. It should be noted that the best fit line shows a non-zero x-intercept (Q₀˜214 J/cm³), indicating that a certain amount of hydration occurs after which the material starts to gain (measurable) strength. However, it is also noted that once strength development begins (Q₀>214 J/cm³), the rate of strength gain is very similar for all the paste mixtures. Overall the linear correlation demonstrated provides a predictive basis to link the progress of reactions to mechanical properties as described in further detail below.

Development of a Prediction Model for the Progress of Hydration Reactions:

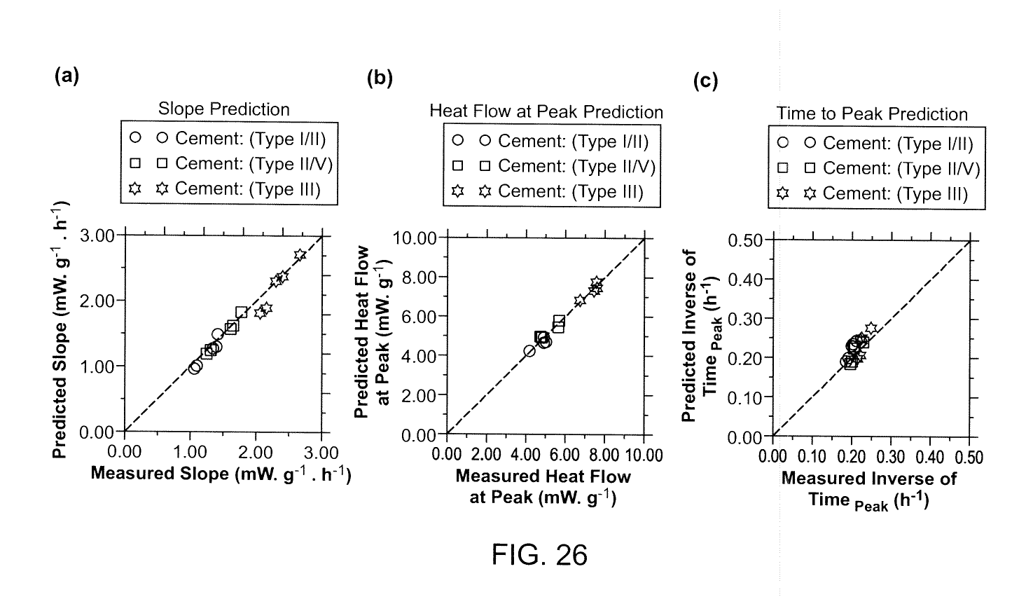

To establish relationships to describe the influence of limestone fineness on the hydration response of cement, calorimetric parameters including the: (a) slope during the acceleration period, (b) heat flow at the main peak, and (c) inverse of time to achieve the heat peak are plotted as a function of the AM (FIG. 25). These relationships are then fitted using separate 4-parameter sigmoid growth functions as shown in Eq. (31) and Table VII:

$$CP ( AM ) = \text{?} [ {C}_{2} + \frac{( {C}_{1} - {C}_{2} )}{exp ( \text{?} )} ] \\ \text{?} \text{indicates text missing or illegible when filed} \qquad ( 31 )$$

where, M_{F,CP}=A·SSA_{c}+B
   where CP is a given calorimetric (effect) parameter, C₁, C₂, C₃, and C₄ are generic fitting constants for each calorimetric parameter, the values of which are listed in Table VII, and AM is the area multiplier (unitless; cause parameter). M_{F,CP} is a multiplication factor (unitless, FIG. 25d) which scales the fitting function (Eq. 31) in relation to the surface area of the cement (m²/kg) and includes A and B as fitting constants pertinent to a given calorimetric parameter (slope during the acceleration period, heat flow at the main peak, or inverse of time to achieve the heat peak).

TABLE VII
Constants used in Calculation of the Calorimetric
Parameters using Eq. (31).

| | Slope | Heat Flow at Peak | Inverse Time to |
|---|---|---|---|
| Parameters | (mW/g_{cement}h) | (mW/g_{cement}) | Peak (/h) |
| C1 | −1.0013 | 2.4800 | 0.0949 |
| C2 | 1.6359 | 5.5871 | 0.2359 |
| C3 | 0.9189 | 0.8715 | 0.3311 |
| C4 | 0.8600 | 1.4567 | 1.5222 |
| A | 0.0040 | 0.0020 | 0.0010 |
| B | −1.1000 | −0.1037 | 0.5672 |

The form of Eq. (31) is generic enough to describe each calorimetric parameter over a wide range of limestone particle sizes and cement replacement levels (AMs). Once quantified for a single cement across a range of replacement levels or AMs, the calorimetric parameters for other cement/limestone combinations can be predicted, with apriori knowledge of the cement fineness (FIG. 25d) and the reaction response of a plain cement paste (see below). This approach is applicable to cements which show broadly similar chemistries (major phase compositions).

The approach used to predict the hydration response applies a family of piecewise linear functions to describe the heat flow response, through the different stages of hydration including: dissolution, induction, acceleration, deceleration, steady state, and so forth. This approach is relativistic in that it uses the: (1) reaction rate curve applicable to the reference plain cement paste for a given cement and (2) calorimetric parameters described using Eq. (31) and shown in FIG. 25 to predict the rate of reactions when the cement content is reduced by replacement with particle size classified limestone. Based on these aspects, while the pre-acceleration regime features are assumed to be similar independent of the cement replacement level, post-induction regime features are described as a function of the AM (fineness and quantity of the cement and limestone in a given mixture). These piecewise linear functions for prediction of heat up to 3 days of hydration are described by Eq. (32).

P_{HF}(t)=M_{HF}(t) for (t<t_{IND,Ref})

P_{HF}(t)=[Slope_{ACC}·t] for (t_{IND,Ref}<t<t_{PEAK})

P_{HF}(t_{PEAK})=[Max[(Slope_{ACC}·t_{PEAK}),(H_{PEAK}))] for (t_{PEAK}<t<t_{PEAK}+3 h)

P_{HF}(t)=[P_{HF}(t)−3.35·Slope_{ACC}·(t−(t_{PEAK}+3))] for (t_{PEAK}+3 h<t and P_{HF}(t)>1.00)

P_{HF}(t)=[1.289−0.017·(t)] for (t≦72 h and 0.00≦P_{HF}(t)≦1.00)

P_{CH}(t)=∫₀^{t}P_{HF}(t)dt for (0≦t≦72 h) (32)

where P_{HF}(t) is the predicted heat flow at a given instant in time (mW/g_{CEM}), M_{HF}(t) is the heat flow measured using isothermal calorimetry (mW/g_{CEM}) for a plain cement paste system for a given cement, t is the reaction time (ranging between 0-72 h), t_{IND,Ref} is the reaction time at which the induction period terminates for the plain cement paste system determined by reverse projection (to the x-axis) of the heat flow response during the acceleration regime (h), t_{PEAK} (h) and H_{PEAK} (mW/g_{CEM}) are the reaction time and magnitude/amplitude corresponding to the main heat peak, Slope_{ACC} is the slope of the heat flow curve during the acceleration regime (mW/g_{CEM}/hour), and P_{CH}(t) is the predicted cumulative heat released due to chemical reactions in a given reaction time period (J/g_{CEM}). Per the equations above, the following assumptions are made: (a) the addition of limestone does not substantially alter the pre-acceleration features of hydration, (b) the reaction maintains its maximum rate for 3 h independent of the level of cement replacement and (c) the reaction decelerates at a fixed rate until a heat flow value of 0.00 mW/g_{CEM} is achieved.

Given the relatively linear evolution of heat after early times (after 72 hours) for typical mixtures, the heat evolved at 7 and 28 days can be estimated with a high level of accuracy by multiplying the 3 day heat value by a constant factor as shown in Eq. (33). For the range of mixtures considered in this example, this multiplication yielded 7 and 28 day heat values with an accuracy greater than about 95%.

P_{CH}(7 days)=[1.20P_{CH}(72 h)]

P_{CH}(28 days)=[1.32P_{CH}(72 h)] (33)

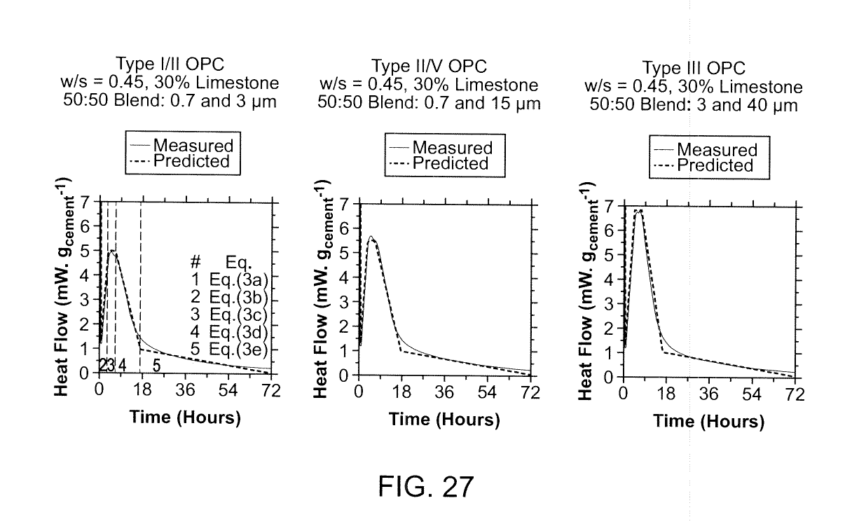

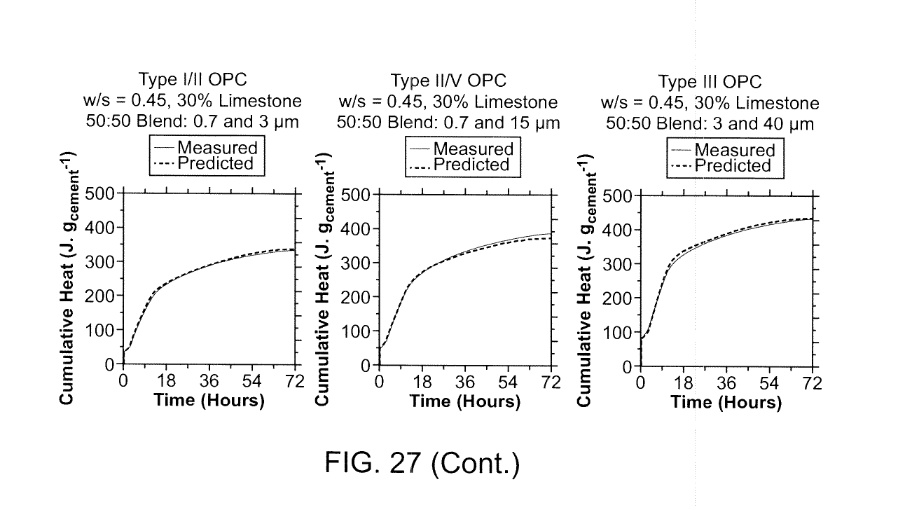

The accuracy and robustness of the prediction model is tested using a variety of mixed systems which remain undefined in the training set as shown in FIG. 26. Here, for a given cement type, and a single cement replacement level by limestone (30%, weight basis), two gradations (powders of differing median particle sizes) of limestone are intermixed in equal parts (e.g., 50%-50% by weight blend of the 0.7 and 3 micron) rather than a single gradation (e.g., 0.7 micron) prior to being used to replace cement in the mixture. It is noted that that a piecewise linear approach is able to very accurately predict the calorimetric parameters and the heat flow and cumulative heat release curves for the mixed systems (as also the other systems defined in the training set, not shown) to describe the evolution of hydration reactions in these materials, as shown in FIG. 26 and FIG. 27.

Modifications in the formulas used can be implemented in the case of substantial changes in the cement chemistry and the use of fillers (compositionally) different from limestone. It is also noted that, while increasing area (either, or both, cement and limestone fineness) does accelerate reactions, for AM≧4, further increase in surface area (filler fineness) may yield reduced benefits. The approach is useful in that, for a relatively broad selection of cement types and limestone fineness, it can describe the evolution of hydration reactions in cementing systems in relation to the cement and filler fineness, and can be used to predict the acceleratory effects of limestone addition on binder hydration reactions—valuable information which could be used to proportion and dial in limestone additions to address aspects of set and strength retardation in low-cement content and cement-replaced formulations.

Estimating Mechanical Property Evolution from the Heat Release Response:

FIG. 24 demonstrated a strongly correlated relationship between the evolution of strength and heat release in a hydrating paste system. It is noted that, for a variety of cements with differing w/c, limestone replacement levels, and limestone particle size distributions, a single linear function can be used to reliably link heat release through hydration (e.g., measured using calorimetry) to compressive strength development. In conjunction with the reaction prediction model described above, a virtual testing model can be implemented to describe the influence of size classified limestone additions, cement fineness, and limestone replacement level on strength development. This approach, which uses as inputs physical properties (SSA_{C} and SSA_{F}) and mixture proportions (w/s, r) of the materials, estimates strength development using Eq. (32) and a single calorimetry measurement of a reference (plain) cement paste. Upon estimation of the cumulative heat release at a sought age, Eq. (34) can be used to estimate strength development in a cementitious mixture as:

S_{Predicted}(t)=0.06·P_{CH-water}(t)−2.110 (34)

Where S_{Predicted}(t) is the predicted compressive strength at a given age (MPa), P_{CH-water}(t) is the predicted cumulative heat flow at a given specimen age (e.g., 1, 3, 7, or 28 days) normalized by the initial water content of the mixture (J/g_{WATER}). It should be noted that this relationship is built on the basis of the linear-fit which best describes the data-cloud shown in FIG. 24.

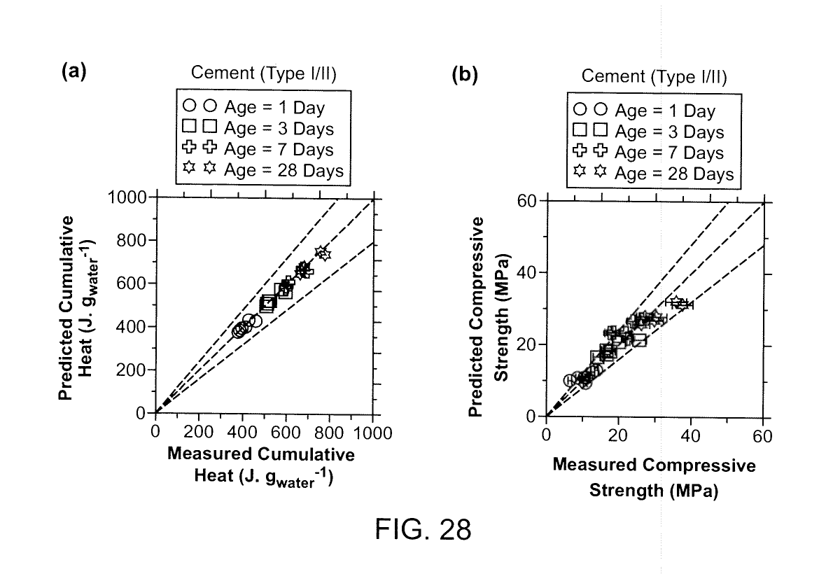

The accuracy and robustness of the strength prediction model was validated for: (1) the mixed systems described above, constituted using two distinct gradations of limestone and (2) w/s=0.45 mixtures in which 15% of the OPC (weight basis) is replaced by limestone powders having a median diameter of 0.7 and 3 μm. A replacement level of 15% (weight basis) achieved by post-blending is specifically chosen, in light of recent regulatory actions in the U.S., which allow OPC replacement (by limestone) at these levels. Procedurally, the predictions are accomplished as follows: first, the cumulative heat release of any given mixture through hydration is calculated at 1, 3, 7, and 28 days using Eqs. (32) and (33) and compared against experiment (FIG. 28a). It is noted that, across a range of cement types, limestone gradations, and OPC replacement levels, highly accurate heat predictions (average error ˜2%) are obtained (see FIGS. 26-28a)). Second, the predicted cumulative heat values (P_{CH-water}) are input into Eq. (34) to predict the evolution of strength (FIG. 28b). Once again, the evolution of compressive strength for a broad range of mixtures can be predicted with an average error of about 12% across all mixtures.

While this example develops the prediction model specifically for limestone-based additions, similar prediction models can be extended to other mineral fillers that are essentially inert at early ages (e.g., Class F fly ash, quartz, and so forth). Refinements can account for different levels of mineral acceleration and intrinsic chemical reactivity (e.g., as being pozzolanic or hydraulic) of different cement replacement agents. The refinements can enhance the accuracy of the prediction at later ages to account for changes in the reaction mechanism or process (e.g., as related to pozzolanic reactions which progress slowly). Additional considerations include: (1) curing (saturated, sealed, or mixed), as curing alters the nature and extent of strength development, more so in the case of water-deficient (low w/c) materials, (2) curing temperature (e.g., curing at ambient (25° C.) versus higher temperatures (e.g., 60° C.)) when microstructural changes can result in less than expected strength, despite similarities in the extent of hydration (heat release), and (3) determinations of cement paste, mortar, or concrete specimens, due to the influences of aggregate volume fraction, gradation and aggregate stiffness, or substantial changes in the cement chemistry (e.g., blended cements).

Conclusions:

This example has described the influence of size classified limestone additions on reaction and property development in cementitious mixtures. The specific influences of limestone fineness, OPC type, and replacement level are quantified by: (1) reaction rate parameters identified using isothermal calorimetry and (2) compressive strength evolution in paste mixtures. First, based on a large database of quantifications (a training set), a model based on piecewise linear functions is developed to estimate the influence of size classified limestone additions on the rate and extent of reactions. Second, correlations between cumulative heat release through hydration and compressive strength development are tapped to identify a “strength-heat master curve” (SHMC). In conjunction with the reaction prediction model, the SHMC sets a basis for estimating the time dependent evolution of strength in paste mixtures composed using a variety of OPCs, for differing limestone gradations, and OPC replacement levels. The robustness of the model is verified using blind tests conducted against mixtures which remain undefined in the training set. The accuracy of these predictions is identified to be on the order of 2% and 12%, for cumulative heat and compressive strength estimations respectively, for timelines ranging from 1 day to 28 days. Overall, the example develops methods to estimate, apriori, the influence of mixture proportions on hardened properties, and makes contributions towards advancing methods of binder formulation and proportioning.

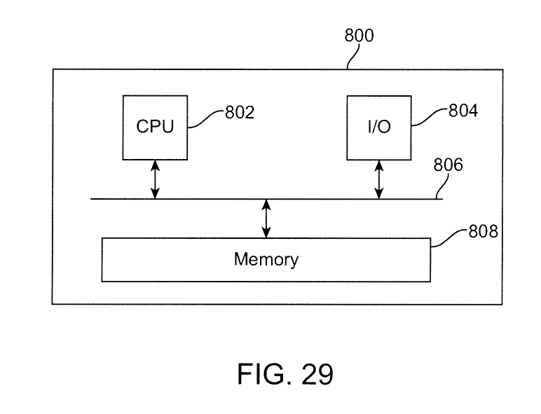

FIG. 29 illustrates a computer 800 configured in accordance with an embodiment of this disclosure. The computer 800 includes a central processing unit (CPU) 802 connected to a bus 806. Input/output (I/O) devices 804 are also connected to the bus 806, and can include a keyboard, mouse, display, and the like. A computer program implementing a tool and a prediction model as described above is stored in a memory 808, which is also connected to the bus 106. A dataset also can be stored in the memory 808, such as in the form of a database.

An embodiment of the disclosure relates to a non-transitory computer-readable storage medium having computer code thereon for performing various computer-implemented operations. The term “computer-readable storage medium” is used herein to include any medium that is capable of storing or encoding a sequence of executable instructions or computer codes for performing the operations, methodologies, and techniques described herein. The media and computer code may be those specially designed and constructed for the purposes of the invention, or they may be of the kind well known and available to those having skill in the computer software arts. Examples of computer-readable storage media include, but are not limited to: magnetic media such as hard disks, floppy disks, and magnetic tape; optical media such as CD-ROMs and holographic devices; magneto-optical media such as floptical disks; and hardware devices that are specially configured to store and execute program code, such as application-specific integrated circuits (ASICs), programmable logic devices (PLDs), and ROM and RAM devices. Examples of computer code include machine code, such as produced by a compiler, and files containing higher-level code that are executed by a computer using an interpreter or a compiler. For example, an embodiment of the disclosure may be implemented using Java, C++, or other object-oriented programming language and development tools. Additional examples of computer code include encrypted code and compressed code. Moreover, an embodiment of the disclosure may be downloaded as a computer program product, which may be transferred from a remote computer (e.g., a server computer) to a requesting computer (e.g., a client computer or a different server computer) via a transmission channel. Another embodiment of the disclosure may be implemented in hardwired circuitry in place of, or in combination with, machine-executable software instructions.

While the invention has been described with reference to the specific embodiments thereof, it should be understood by those skilled in the art that various changes may be made and equivalents may be substituted without departing from the true spirit and scope of the invention as defined by the appended claims. In addition, many modifications may be made to adapt a particular situation, material, composition of matter, method, operation or operations, to the objective, spirit and scope of the invention. All such modifications are intended to be within the scope of the claims appended hereto. In particular, while certain methods may have been described with reference to particular operations performed in a particular order, it will be understood that these operations may be combined, sub-divided, or re-ordered to form an equivalent method without departing from the teachings of the invention. Accordingly, unless specifically indicated herein, the order and grouping of the operations is not a limitation of the invention.

What is claimed is:

1. A non-transitory computer-readable storage medium, comprising executable instructions to:
receive user input characterizing a mixture of a cement and a mineral addition, the user input corresponding to at least one of: (a) a size characteristic of the cement; (b) a size characteristic of the mineral addition; and (c) a replacement level of the cement by the mineral addition in the mixture;
based on the user input, derive a predicted cumulated heat released by the mixture through hydration for a reaction time period; and
based on the predicted cumulated heat released, derive a predicted mechanical property of the mixture at an age corresponding to the reaction time period.

2. The non-transitory computer-readable storage medium of claim 1, wherein the executable instructions to derive the predicted cumulated heat released include executable instructions to:
based on the user input, derive an area multiplier characterizing a change in solid surface area resulting from replacement of the cement by the mineral addition in the mixture.

3. The non-transitory computer-readable storage medium of claim 2, wherein the size characteristic of the cement and the size characteristic of the mineral addition correspond to a particle size distribution of the cement and a particle size distribution of the mineral addition, respectively, and the executable instructions to derive the area multiplier include executable instructions to:
based on the particle size distribution of the cement and the particle size distribution of the mineral addition, derive a specific surface area of the cement and a specific surface area of the mineral addition.

4. The non-transitory computer-readable storage medium of claim 2, wherein the executable instructions to derive the predicted cumulated heat released include executable instructions to:
based on the area multiplier, derive calorimetric parameters characterizing a predicted heat flow response of the mixture through hydration.

5. The non-transitory computer-readable storage medium of claim 4, wherein the calorimetric parameters correspond to at least one of:
a slope during an acceleration time period;
a heat flow at a main peak; and
a time to reach the main peak.

6. The non-transitory computer-readable storage medium of claim 4, wherein the executable instructions to derive the predicted cumulated heat released include executable instructions to:
integrate the predicted heat flow response over at least a portion of the reaction time period.

7. The non-transitory computer-readable storage medium of claim 1, wherein the mineral addition is limestone.

8. The non-transitory computer-readable storage medium of claim 1, wherein the predicted mechanical property is a predicted compressive strength of the mixture.

9. A non-transitory computer-readable storage medium, comprising executable instructions to:
provide a prediction model relating (a) a size characteristic of a cement, (b) a size characteristic of a mineral addition, (c) a replacement level of the cement by the mineral addition in a cementitious mixture, and (d) a mechanical property of the cementitious mixture;
receive user input corresponding to a desired value of the mechanical property; and
based on the prediction model, identify a candidate cementitious mixture having a predicted value of the mechanical property that matches the desired value of the mechanical property.

10. The non-transitory computer-readable storage medium of claim 9, wherein the executable instructions to identify the candidate cementitious mixture include executable instructions to:
identify a candidate size characteristic of the cement to yield the predicted value of the mechanical property that matches the desired value of the mechanical property.

11. The non-transitory computer-readable storage medium of claim 10, wherein the executable instructions to identify the candidate cementitious mixture include executable instructions to:
based on the candidate size characteristic of the cement, derive a predicted cumulated heat released by the candidate cementitious mixture through hydration for a reaction time period;
based on the predicted cumulated heat released, derive the predicted value of the mechanical property of the candidate cementitious mixture at an age corresponding to the reaction time period; and
compare the predicted value of the mechanical property with the desired value of the mechanical property.

12. The non-transitory computer-readable storage medium of claim 9, wherein the executable instructions to identify the candidate cementitious mixture include executable instructions to:
identify a candidate size characteristic of the mineral addition to yield the predicted value of the mechanical property that matches the desired value of the mechanical property.

13. The non-transitory computer-readable storage medium of claim 12, wherein the executable instructions to identify the candidate cementitious mixture include executable instructions to:
based on the candidate size characteristic of the mineral addition, derive a predicted cumulated heat released by the candidate cementitious mixture through hydration for a reaction time period;
based on the predicted cumulated heat released, derive the predicted value of the mechanical property of the candidate cementitious mixture at an age corresponding to the reaction time period; and
compare the predicted value of the mechanical property with the desired value of the mechanical property.

14. The non-transitory computer-readable storage medium of claim 9, wherein the executable instructions to identify the candidate cementitious mixture include executable instructions to:
identify a candidate replacement level of the cement by the mineral addition to yield the predicted value of the mechanical property that matches the desired value of the mechanical property.

15. The non-transitory computer-readable storage medium of claim 14, wherein the executable instructions to identify the candidate cementitious mixture include executable instructions to:
based on the candidate replacement level of the cement by the mineral addition, derive a predicted cumulated heat released by the candidate cementitious mixture through hydration for a reaction time period;
based on the predicted cumulated heat released, derive the predicted value of the mechanical property of the candidate cementitious mixture at an age corresponding to the reaction time period; and
compare the predicted value of the mechanical property with the desired value of the mechanical property.

16. The non-transitory computer-readable storage medium of claim 9, wherein the cement is Portland cement.

17. The non-transitory computer-readable storage medium of claim 9, wherein the mineral addition is selected from at least one of limestone, quartz, Fly ash, and silica fume.
